(19) United States
(12) Patent Application Publication
(10) Pub. No.: US 2011/0060197 A1
(43) Pub. Date: Mar. 10, 2011

(54) NEAR INFRARED SPECTROPHOTOMETRY WITH ENHANCED SIGNAL TO NOISE PERFORMANCE

(75) Inventors: Wei ZHANG, Union City, CA (US); Shih-Ping Wang, Los Altos, CA (US); Shuoming Zhou, Cupertino, CA (US); Zengpin Yu, Palo Alto, CA (US)

(73) Assignee: O2 MEDTECH, INC., Los Altos, CA (US)

(21) Appl. No.: 12/832,603

(22) Filed: Jul. 8, 2010

Related U.S. Application Data
(63) Continuation-in-part of application No. 12/826,218, filed on Jun. 29, 2010.
(60) Provisional application No. 61/224,684, filed on Jul. 10, 2009.
(60) Provisional application No. 61/222,099, filed on Jun. 30, 2009.
(60) Provisional application No. 61/255,851, filed on Oct. 28, 2009.

(51) Int. Cl. A61B 5/1455

(52) U.S. Cl. 600/310

(57) ABSTRACT

Methods, systems, and related computer program products for the non-invasive spectrophotometric monitoring of an optical property of a tissue volume are described. Multiple optical signals having different modulation frequencies are introduced into the tissue volume simultaneously and on a continuous basis throughout the monitoring session. Multiple optical signal portions incident upon each of a plurality of optical detectors are detected and separated based on their modulation frequency. Amplitude and phase signals corresponding to each optical signal portion are extracted and processed to determine the optical property of the tissue volume. In one preferred embodiment, a first optical detector includes an aperture having a central area, a first edge positioned nearer to a first optical source than the central area, and a second edge positioned farther from the first optical source than the central area. The first and second edges are each curved concavely toward the first optical source.

### CROSS-REFERENCE TO RELATED APPLICATIONS

This patent application claims the benefit of U.S. Provisional Ser. No. 61/224,684, filed Jul. 10, 2009. This patent application is a continuation-in-part of U.S. Ser. No. 12/826,218, filed Jun. 29, 2010 (Atty. Dkt. 6949/81720), which claims the benefit of U.S. Provisional Ser. No. 61/222,099, filed Jun. 30, 2009, and which also claims the benefit of U.S. Provisional Ser. No. 61/255,851, filed Oct. 28, 2009. Each of the above-referenced patent applications is incorporated by reference herein.

### FIELD

This patent specification relates to the non-invasive monitoring of a physiological condition of a patient using information from non-invasive near-infrared (NIR) optical scans. More particularly, this patent specification relates to systems, methods, and related computer program products for improving signal to noise performance in the non-invasive near-infrared spectrophotometric (NIRS) monitoring of chromophore levels in biological tissue.

### BACKGROUND AND SUMMARY

The use of near-infrared (NIR) light as a basis for the measurement of biological properties or conditions in living tissue is particularly appealing because of its relative safety as compared, for example, to the use of ionizing radiation. Various techniques have been proposed for non-invasive NIR spectroscopy or NIR spectrophotometry (NIRS) of biological tissue. Generally speaking, these techniques are directed to detecting the concentrations of one or more chromophores in the biological tissue, such as blood hemoglobin in oxygenated (HbO) and deoxygenated (Hb) states.

As used herein, NIR tissue oxygenation level monitoring refers to the introduction of NIR radiation (e.g., in the 500-2000 nm range) into a tissue volume and the processing of received NIR radiation migrating outward from the tissue volume to generate at least one metric indicative of oxygenation level(s) in the tissue. One example of an oxygenation level metric is oxygen saturation [SO₂], which refers to the fraction or percentage of total hemoglobin [HbT] that is oxygenated hemoglobin [HbO]. NIRS-based oxygen saturation readings can be classified as “relative” in nature (i.e., presented only in terms of their change over time) or can be “absolute” in nature (i.e., computed from absolute concentrations of [HbO] and [HbT] in units of grams per deciliter (g/dl) or equivalent).

NIR cerebral oxygenation level monitoring, which refers to the transcranial introduction of NIR radiation into the intracranial compartment and the processing of received NIR radiation migrating outward therefrom to generate at least one metric indicative of oxygenation level(s) in the brain, represents one particularly important type NIR tissue oxygenation level monitoring. One exemplary need for reliable determination of oxygen saturation levels in the human brain arises in the context of the millions of surgical procedures performed under general anesthesia every year. One statistic recited in U.S. Pat. No. 5,902,235 is that at least 2,000 patients die each year in the United States alone due to anesthetic accidents, while numerous other such incidents result in at least some amount of brain damage. Certain surgical procedures, particularly of a neurological, cardiac or vascular nature, may require induced low blood flow or pressure conditions, which inevitably involves the potential of insufficient oxygen delivery to the brain. Many surgical procedures also involve the possibility that a blood clot or other clottable material can break free, or otherwise get introduced into the bloodstream, and travel to the brain to cause a localized or widespread ischemic event therein. At the same time, the brain is highly intolerant to oxygen deprivation, and brain cells will die (become infarcted) within a few minutes if not sufficiently oxygenated. Accordingly, the availability of immediate, accurate and reliable information concerning brain oxygenation levels is of critical importance to anesthesiologists and surgeons, as well as other involved medical practitioners.

Pulse oximetry, in which infrared sources and detectors are placed across a thin part of the patient's anatomy such as a fingertip or earlobe, has arisen as a standard of care for all operating room procedures. However, pulse oximetry provides only a general measure of blood oxygenation as represented by the blood passing by the fingertip or earlobe sensor, and does not provide a measure of oxygen levels in vital organs such as the brain. In this sense, the surgeons in the operating room essentially “fly blind” with respect to brain oxygenation levels, which can be a major source of risk for patients (e.g., stroke) as well as a major source of cost and liability issues for hospitals and medical insurers.

Valid NIR cerebral oxygenation level readings provide crucial monitoring data for the surgeon and other attending medical personnel, providing more direct data on brain oxygenation levels than pulse oximeters while being just as safe and non-invasive as pulse oximeters. Generally speaking, such systems involve the attachment of an NIR probe patch, or multiple such NIR probe patches, to the forehead and/or other available skin surface of the head. Each NIR probe patch usually comprises one or more NIR optical source ports for introducing NIR radiation into the cerebral tissue and one or more NIR optical receiver ports for detecting NIR radiation that has migrated through at least a portion of the cerebral tissue. One or more oxygenation level metrics are then provided on a viewable display in a digital readout and/or graphical format.

One issue that arises in NIR cerebral oximetry is the need for substantial signal penetration depth in order to obtain useful readings for the brain tissue itself, which lies beneath several intervening layers including the skin, scalp, skull, dura, and cerebrospinal fluid (CSF) layers. According to one thumbnail estimate provided in U.S. Pat. No. 5,853,370, which is incorporated by reference herein, the average penetration depth for a NIRS source-detector pair is about one-half of the lateral separation between the source and the detector. Thus, to acquire meaningful readings for brain tissue at a depth of about 3 cm from the skin surface, the source-detector distance needs to be about 6 cm. However, due to the high degree of signal degradation involved, such relatively large source-detector distances have not been provided in known commercially available NIR cerebral oximeters. It would be desirable to provide an NIR cerebral oximeter with improved signal-to-noise performance in order to accommodate such relatively large source-detector distances. Furthermore, improved signal to noise performance would also increase the accuracy and/or reliability of the readings provided for more closely-spaced source-detector pairs. Other issues arise as would be apparent to one skilled in the art upon reading the present disclosure.

It is to be appreciated that although one or more preferred embodiments is detailed hereinbelow in the particular context of NIR cerebral oxygenation level monitoring (NIR cerebral oximetry), the present teachings are readily applicable to the non-invasive spectrophotometric monitoring of any of a variety of different body parts including, but not limited to, the kidney, lung, liver, arm, leg, neck, etc., and furthermore are applicable for the monitoring of any of a variety of different chromophore types therein.

Provided according to one or more preferred embodiments are methods, systems, and related computer program products for non-invasive spectrophotometric monitoring of an optical property of a tissue volume during a patient monitoring session. A plurality of optical sources and a plurality of optical detectors are secured to a surface of the tissue volume. The plurality of optical sources are operated to introduce, simultaneously and on a continuous basis throughout the patient monitoring session, a plurality of optical signals into the tissue volume. Preferably, each of the optical signals has a modulation frequency different than that of each other optical signal, and any two of the optical signals that are introduced from a same one of the optical sources are at different optical wavelengths. The plurality of optical detectors are each operated to detect, simultaneously and on a continuous basis throughout the monitoring session, a portion of each of the optical signals that has propagated thereto, and each of the detected optical signal portions is processed to derive an amplitude signal and a phase signal associated therewith. The derived amplitude signals and phase signals associated with the detected optical signal portions are then processed to determine the optical property of the tissue volume.

Also provided is an apparatus for non-invasive spectrophotometric monitoring of an optical property of a tissue volume of a patient during a patient monitoring session. The apparatus comprises a probe patch wearable on a surface of the tissue volume, the probe patch comprising a plurality of optical sources and a plurality of optical detectors. The probe patch is configured to maintain each of the optical sources and each of the optical detectors in secured contact with the surface of the tissue volume throughout the patient monitoring session. The apparatus further comprises a source controller coupled to each of the plurality of optical sources, the source controller being configured to cause the plurality of optical sources to introduce, simultaneously and on a continuous basis throughout the patient monitoring session, a plurality of optical signals into the tissue volume, each optical signal having a modulation frequency different than that of each other optical signal, wherein any two of the optical signals that are introduced from a same one of the optical sources are at different optical wavelengths. The apparatus further comprises a detector controller coupled to each of the plurality of optical detectors, the detector controller being configured to cause each of the plurality of optical detectors to detect, simultaneously and on a continuous basis throughout the monitoring session, a portion of each of the optical signals that has propagated thereto. The apparatus further comprises at least one processor configured to process each of the detected optical signal portions to derive an amplitude signal and a phase signal associated therewith, the at least one processor being further configured to process the amplitude signals and phase signals associated with the detected optical signal portions to determine the optical property of the tissue volume.

Also provided is an apparatus for non-invasive spectrophotometric monitoring of an optical property of a tissue volume of a patient during a patient monitoring session, comprising a probe patch wearable on a surface of the tissue volume of the patient. A first optical source and a first optical detector are disposed on the probe patch. The probe patch is configured to maintain each of the first optical source and the first optical detector in secured contact with the surface of the tissue volume throughout the patient monitoring session. The first optical detector includes a first aperture formed in a tissue-facing surface of the wearable patch. The first aperture includes a central area, a first edge positioned nearer to the first optical source than the central area, and a second edge positioned farther from the first optical source than the central area. Preferably, the first and second edges of the first aperture are each curved concavely toward the first optical source.

Among other advantages, non-invasive near-infrared spectrophotometric monitoring according to one or more of the preferred embodiments provides for improved signal to noise performance. Among other advantages, the improved signal to noise performance provides an ability to increase penetration depths in the non-invasive NIRS monitoring of crucial deep-layer tissue structures including, but not limited to, the human brain.

### BRIEF DESCRIPTION OF THE DRAWINGS

FIG. 1 illustrates a prior art bilateral cerebral spectrophotometric monitoring system;

FIGS. 2A-2C illustrate a prior art slope method used in spectrophotometric monitoring;

FIGS. 3A-3C illustrate equations used in particular prior art phase modulated spectrophotometric (PMS) and continuous wave (CW) spectrophotometric monitoring scenarios;

FIG. 4 illustrates a prior art arrangement of non-ideal optical sources and detectors on a probe patch;

FIG. 5 illustrates an intensity-based slope computation based on a prior art symmetric source-detector layout;

FIG. 6 illustrates an intensity-based slope computation based on a prior art symmetric source-detector layout;

FIG. 7A illustrates a near-infrared spectrophotometric (NIR) cerebral oximeter according to a preferred embodiment;

FIG. 7B-1 illustrates an NIR probe patch according to a preferred embodiment;

FIG. 7B-2 illustrates exemplary dimensions associated with the NIR probe patch of FIG. 7B-1;

FIG. 7C illustrates an NIR probe patch according to a preferred embodiment;

FIGS. 7D-1 and 7D-2 illustrate an NIR probe patch according to a preferred embodiment;

FIG. 7E illustrates the NIR probe patch of FIG. 7D-1 as applied to a surface of a biological volume according to a preferred embodiment;

FIG. 7F illustrates dual instances of the NIR probe patch of FIG. 7D-1 as applied to a forehead of a patient for bilateral cerebral oximetry according to a preferred embodiment;

FIG. 8A illustrates near-infrared spectrophotometric (NIRS) monitoring of a biological volume of a patient according to a preferred embodiment;

FIG. 8B illustrates an alternative version of the probe patch illustrated in FIG. 8A according to a preferred embodiment;

FIGS. 9A-9C illustrate equations for adapting a slope method for NIRS monitoring of a biological volume according to a preferred embodiment;

FIG. 10 illustrates NIRS monitoring of a biological volume of a patient according to a preferred embodiment;

FIG. 11 illustrates NIRS monitoring of a tissue volume in which there is a relatively low duty cycle for any particular source/wavelength pair;

FIG. 12 illustrates NIRS monitoring of a tissue volume of a patient according to a preferred embodiment;

FIG. 13 illustrates a block diagram of signal processing circuitry for use with a detector of a NIRS monitoring system according to a preferred embodiment;

FIG. 14 illustrates NIRS monitoring of a tissue volume of a patient according to a preferred embodiment;

FIGS. 15A-15B illustrate an NIR probe patch including curved-edge detection apertures according to a preferred embodiment;

FIG. 16 illustrates an NIR probe patch including curved-edge detection apertures according to a preferred embodiment; and

FIG. 17 illustrates NIRS monitoring of a tissue volume of a patient according to a preferred embodiment.

### DETAILED DESCRIPTION

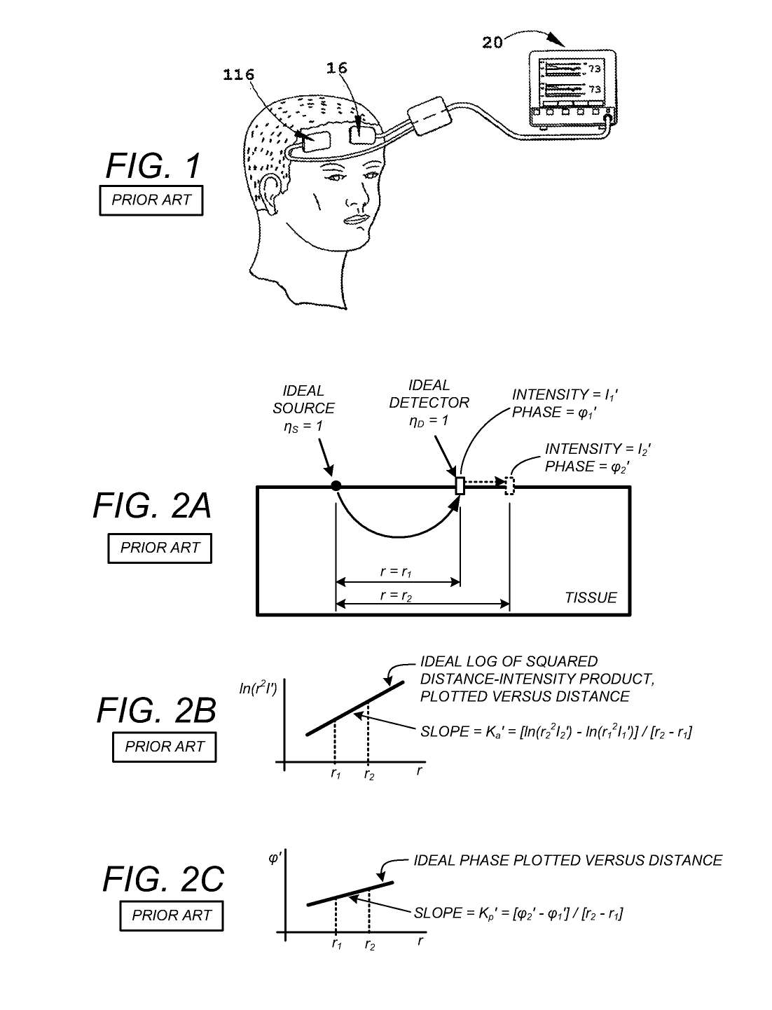

FIG. 1 illustrates a prior art proposal for a bilateral monitoring system in which two NIR probe patches 16 and 116 are placed on the forehead of the patient. The prior art proposal of FIG. 1 is further described in U.S. Pat. No. 6,615,065, which is incorporated by reference herein. Separate readings for the left and right sides of the brain are acquired and displayed separately on an output display 20. As illustrated in the proposal of FIG. 1, NIR probe patches are often placed on the forehead of the patient. The forehead represents a generally desirable region for attaching NIR probe patches, for at least the reason that the forehead is generally free of hair follicles. Even for a smoothly shaved head, the presence of hair follicles can introduce substantial amounts of noise and other interference into the NIR signals.

However, the use in FIG. 1 of two separate NIR probe patches on the forehead is antagonistic to an even more important goal of NIR cerebral oximetry, which is to obtain “deep” readings that are relevant to the brain tissue, rather than to the intervening skin, scalp, skull, dura, and cerebrospinal fluid (CSF) tissue. According to one thumbnail estimate provided in U.S. Pat. No. 5,853,370, which is incorporated by reference herein, the average penetration depth for a NIRS source-detector pair is about one-half of the lateral separation between the source and the detector. Because the source and the detector for any particular source-detector pair are required to be present on the same NIR probe patch (due to the need for precise, predetermined source-detector separation distances), the maximum source-detector separation distance for the prior art proposal of FIG. 1 is limited by the spatial extent of each individual NIR probe patch 16 and 116. Moreover, the use in FIG. 1 of two separate NIR probe patches on the forehead also brings about the need for left-right duplication of multiple source-detector pairs in order to obviate source intensity differences, detector efficiency differences, and skin coupling efficiency differences among the sources and detectors 116.

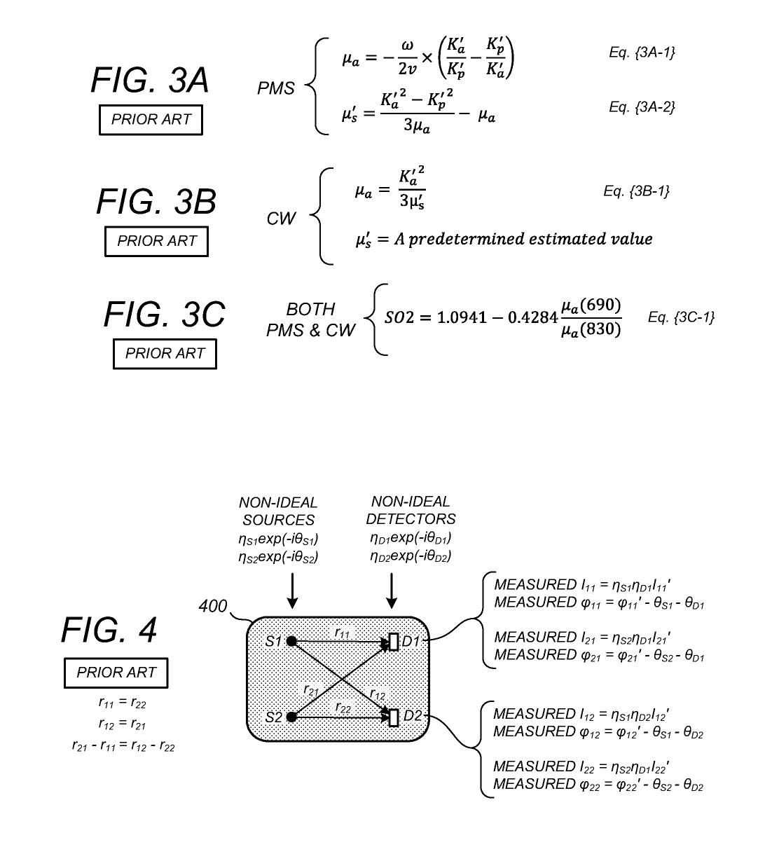

FIGS. 2A-2C illustrate a prior art slope method used in spectrophotometric monitoring. FIGS. 3A-3C illustrate equations used in particular prior art phase modulated spectrophotometric (PMS) and continuous wave (CW) spectrophotometric monitoring scenarios. Summarized in FIGS. 2A-2C and 3A-3C is the well-accepted “slope method” for computing tissue oxygenation levels (see, e.g., Fantini, Franceschini, and Gratton, “Semi-Infinite-Geometry Boundary Problem For Light Migration In Highly Scattering Media: A Frequency-Domain Study In The Diffusion Approximation,” J. Opt. Soc. Am. B, Vol. 11, pp. 2128-38 (1994) and Fantini, Hueber, and Franceschini, et. al., “Non-Invasive Optical Monitoring of the Newborn Piglet Brain Using Continuous-Wave and Frequency-Domain Spectroscopy,” Phys. Med. Biol., Vol. 44, pp. 1543-1563 (1999), each of which is incorporated by reference herein), while FIGS. 4-6 set forth one known method (see, e.g., U.S. Pat. No. 6,078,833, which is incorporated by reference herein) for using multiple source-detector pairs positioned over a common region to obviate source intensity differences, detector efficiency differences, and skin coupling efficiency differences among the sources and detectors.

Notationally, the prime symbol (′) is used to denote ideal intensities (I′) and ideal phases (φ′) that would result from ideal sources and ideal detectors (including ideal skin coupling), as well as ideal slopes (K′) of any plotted functions based on those ideal intensities and phases. In contrast, non-primed versions of those quantities refer to the physically measured versions of those values in the real world, and are termed herein measured intensities (I) and measured phases (φ), as well as measured slopes (K) of the plotted functions based on the measured intensities and measured phases. For PMS (phase modulated spectrophotometry) systems, also termed frequency domain spectrophotometry systems, the basis of the slope method is that for any particular NIR radiation wavelength, a plot of log (r²l′) versus r (where r is the source-detector distance) (FIG. 2B) has a relatively constant slope Kₐ′ over an appreciably useful range of distances, a plot of φ′ versus r (FIG. 2C) also has a relatively constant slope Kₚ′ over an appreciably useful range of distances, and the values of Kₐ′ and Kₚ′ can be used to compute the absorption coefficient μₐ (FIG. 3A, Eq. {3A-1}) and the effective or reduced scattering coefficient μₛ′ (FIG. 3A, Eq. {3A-2}) for that NIR radiation wavelength, where ω is the angular frequency corresponding to the source intensity modulation and v is the speed of light in the tissue. For CW (continuous wave) spectrophotometry systems in which there is no high-frequency modulation or phase measurements, the value of Kₐ′ can be used to compute the absorption coefficient μₐ (FIG. 3B, Eq. {3B-1}) for that NIR radiation wavelength using a fixed estimate of the effective scattering coefficient μₛ′. Based on the absorption coefficient μₐ for multiple NIR wavelengths (on opposite sides of the isosbestic wavelength for oxygenated and deoxygenated hemoglobin) the oxygenated hemoglobin saturation value SO2 is then readily determined, with {Eq. 3C-1} setting forth the formula for the particular NIR wavelengths of 680 nm and 830 nm. Generally speaking, the SO2 reading for the PMS-based measurements can be characterized as an absolute percentage value, whereas the SO2 reading for CW measurements should be taken only as a relative value over time.

As would be readily understood by a person skilled in the art in view of the present disclosure, the term “intensity” (as well as the equation variable “I” in the accompanying drawings) as used herein in the context of a PMS system refers to the amplitude of the AC component of the intensity waveform. Thus, without loss of generality, the terms “amplitude” and “intensity” may be used interchangeably herein to refer to the amplitude of the AC component of the intensity waveform (see, e.g., the Fantini 1999 article, supra, at Section 2.3 thereof).

FIG. 4 illustrates a prior art arrangement of non-ideal optical sources and detectors on a probe patch. As illustrated in FIG. 4, a non-ideal source S can be modeled as an ideal source as modified by a complex coefficient ηₛexp(−iθ_{S}), which is termed herein the source intensity/coupling coefficient. For simplicity of nomenclature, although the magnitude ηₛ is more generally associated with variations in both source intensity and skin coupling, the magnitude ηₛ is simply referenced herein as “source coupling efficiency.” The phase term θₛ is referred to herein as the “source phase error.” Likewise, as illustrated in FIG. 4, a non-ideal detector D can be modeled as an ideal detector as modified by a complex coefficient η_{D}exp(−iθ_{D}), which is termed herein the detector sensitivity/coupling coefficient. For simplicity of nomenclature, although the magnitude η_{D} is more generally associated with variations in both detector sensitivity and skin coupling, the magnitude η_{D} is simply referenced herein as “detector coupling efficiency.” The phase term θ_{D} is referred to herein as the “detector phase error.”

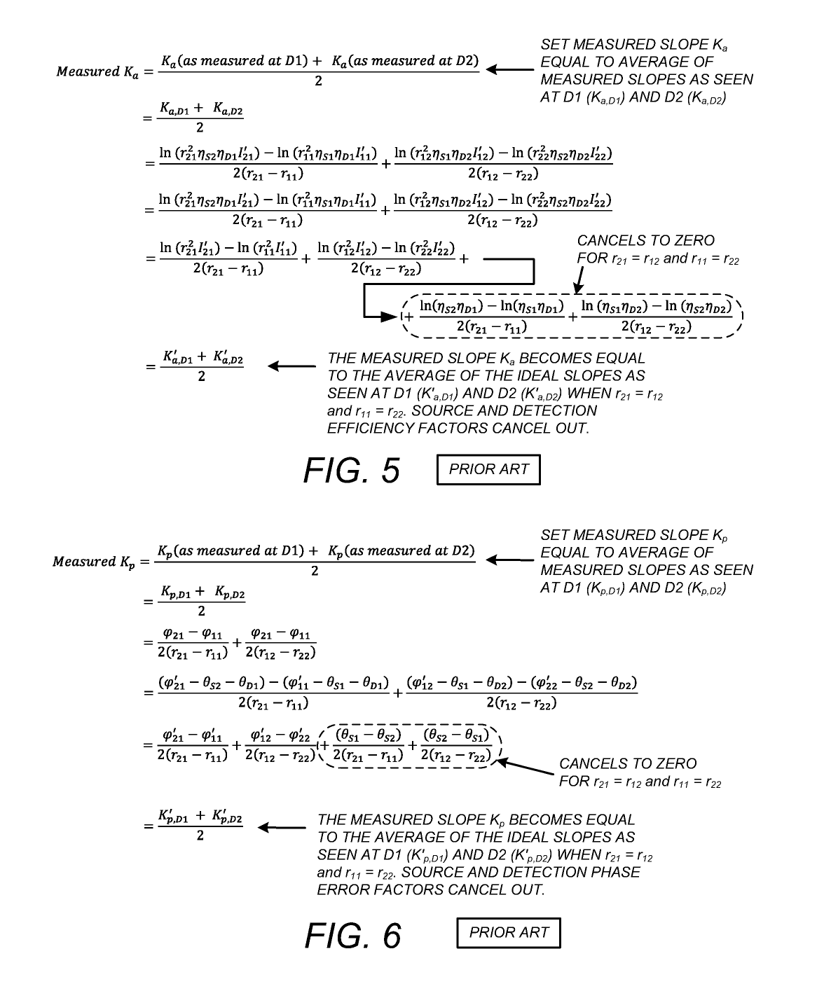

FIG. 5 illustrates an intensity-based slope computation based on a symmetric source-detector layout according to the prior art. FIG. 6 illustrates an intensity-based slope computation based on a symmetric source-detector layout according to the prior art. For simplicity and clarity of explanation, the more general case of PMS modulation is detailed further herein, with it being understood that CW methods would be analogous except with omitted phase factors and omitted phase-related slope computations. In the event that a real-world source 51 (and real-world source-skin coupling) was used and two real-world detectors D1 and D2 (with real-world detector-skin coupling) were positioned at r1 and r2, respectively, in the configuration of FIG. 2A, it could readily be shown that the values of μₐ and μₛ′ would include unknown coupling efficiency and phase error factors in addition to the known measured intensities I₁₂ and I₂₂. Because the coupling efficiency and phase error factors are unknown, the values of μₐ and μₛ′ would either be non-determinable, or else broad assumptions regarding coupling efficiency and phase error factors would need to be made. However, as summarized in FIGS. 4-6 and described further in U.S. Pat. No. 6,078,833, supra, the presence of different coupling efficiencies can be obviated by (i) adding a second source S2, (ii) positioning the two sources S1, S2 and two detectors D1, D2 in a symmetric relationship such that r₂₁=r₁₂ and r₁₁=r₂₂, (iii) computing a first measured slope factor K_{a,D1} representing the slope factor of FIG. 2B for the underlying tissue as “seen” by detector D1, (iv) computing a second measured slope factor K_{a,D2} representing the slope factor of FIG. 2B for that same underlying tissue as “seen” by detector D2, and (v) computing an overall measured slope Kₐ as the arithmetic average of K_{a,D1} and K_{a,D2}. As illustrated in FIG. 5, the coupling efficiencies cancel out such that the measured Kₐ becomes equal to the average of the ideal slopes K′_{a,D1} and K′_{a,D2}, which is tantamount to an overall ideal slope K′ₐ. As illustrated in FIG. 6, the presence of different phase error factors is similarly obviated when r₂₁=r₁₂ and r₁₁=r₂₂, the phase error factors canceling and the overall measured phase slope Kₚ becoming equal to the average of the ideal slopes K′_{p,D1} and K′_{p,D2}, which is tantamount to an overall ideal slope K′ₚ. The resultant values of μₐ, μₛ′, and SO2 are thus independent of the coupling efficiencies and phase error factors, which is indeed a desirable result.

However, as mentioned above, in order for the system of FIG. 1 to achieve this desirable result (i.e., the obviation of source intensity differences, detector efficiency differences, and skin coupling efficiency differences) it is required that each of the left and right NIR probe patches contain a dual arrangement (see FIG. 4) of source-detector pairs for each source-detector separation distance of interest. For a single source-detector separation distance, a 2×2 arrangement (two sources, two detectors, see FIG. 4) is required for each NIR probe patch, thereby requiring a total of eight elements (four sources and four detectors) for the bilateral system. For two source-detector separation distances (for example, a “near” separation distance and a “far” separation distance), a 2×4 arrangement (two sources and four detectors, or four sources and two detectors) is required for each NIR probe patch, thereby requiring a total of twelve elements (four sources and eight detectors, or eight sources and four detectors) for the bilateral system. For three source-detector separation distances (for example, a “near” separation distance, a “mid-range” separation distance, and a “far” separation distance), a 2×6 arrangement (two sources and six detectors, or six sources and two detectors) is required for each NIR probe patch, thereby requiring a total of sixteen elements (four sources and twelve detectors, or twelve sources and four detectors) for the bilateral system. In general, for “N” distinct source-detector separation distances, a (2N+2) arrangement is required for each NIR probe patch, thereby requiring a total of 2(2N+2)=4(N+1) elements for the bilateral system.

Provided according to one preferred embodiment is an NIR cerebral oximeter comprising a unitary across-the-forehead (ATF) patch configured and dimensioned to cover both the left and right sides of the forehead simultaneously, the ATF patch comprising a lateral distribution of NIR sources and detectors including either (i) a plurality of centrally located sources and at least one detector near each of the left and right ends, or (ii) a plurality of centrally located detectors and at least one source near each of the left and right ends, wherein each of the centrally located sources or detectors is used in determining each of (i) an overall chromophore level applicable for the combined left and right sides of the brain, (ii) (ii) a left-side chromophore level separately applicable for the left side of the brain, and (iii) a right-side chromophore level separately applicable for the right side of the brain. While one or more preferred embodiments is described in terms of an across-the-forehead patch for monitoring the left and right brain hemispheres simultaneously, it is to be appreciated that the present teachings further encompass a wide variety of different probe patches capable of simultaneous monitoring of two subregions of tissue that are at least partially non-overlapping, and that the ATF forehead represents but one particularly useful example. Thus, for example, there could be provided in accordance with another preferred embodiment a user-wearable probe patch for monitoring a single kidney, where the first subregion corresponds primarily to an upper part of the kidney and the second subregion corresponds primarily to a lower part of the kidney. As another example, there could be provided in accordance with another preferred embodiment a user-wearable probe patch for monitoring both kidneys, where the first subregion corresponds primarily to a left kidney and the second subregion corresponds primarily to a right kidney.

Also provided according to a preferred embodiment is an algorithm for bilateral chromophore level monitoring based on measurements acquired using the ATF patch sources and detectors, wherein the bilateral chromophore levels are computed in a manner that obviates any coupling efficiency differences or phase error differences among the different sources and detectors, subject only to certain relaxed time-invariance assumptions for the centrally located sources or detectors (specifically, that they exhibit a constant coupling efficiency ratio and a constant phase error difference between them during the monitoring session). Advantageously, because each of the centrally located sources or detectors is involved in the individual monitoring of each of the left and right sides, bilateral monitoring is provided using a reduced number of elements as compared to the use of two separate forehead patches. Advantageously, the spatial geometry of the source/detector elements on the ATF patch provides for increased source-detector separation so that deeper penetration depths into the brain can be achieved in comparison to the use of two separate forehead patches.

As used herein, the term or subscript “whole” is used to refer to a measurement or output reading that is applicable for the combined left and right side tissue of the brain. As will be understood by a person skilled in the art, the terms “whole brain,” “left side of the brain,” and “right side of the brain” as used herein, and unless otherwise stated, refer to those portions that are forward in the skull cavity toward the forehead and reachable by a relevant portion of the NIR radiation that has been introduced into the forehead. The unitary across-the-forehead (ATF) patch can alternatively be termed a whole-forehead patch, cross-forehead patch, or total-forehead patch. Preferably, PMS (phase modulated spectrophotometry) methods are used in conjunction with the ATF sources and detectors such that the absorption coefficient and effective scattering coefficient are each computed for each of a plurality of NIR wavelengths, and absolute SO2 values are provided. However, the preferred embodiments described herein can readily be applied in CW (continuous wave) systems. For simplicity and clarity of explanation, the more general case of PMS modulation is detailed further herein.

It has been found that accurate, clinically useful, absolute, reduced source/detector bilateral SO2 monitoring based on an ATF patch according to one or more of the preferred embodiments can be achieved based on certain clinically reasonable usage and parameter assumptions. A first assumption is that there is a generally quiescent time period at the beginning of a monitoring session in which the whole brain, including both the left and right sides together, can be considered to have a generally uniform SO2 value. This assumption is particularly realistic and useful for exemplary scenarios such as surgery, in which it can be assumed that no blood clots have broken free and traveled to the brain prior to the surgery (for example), and it which case it will be particularly useful to localize which side of the brain a clot is affecting if such an event occurs during the surgery.

A second assumption is that the coupling efficiencies and phase errors of the centrally located sources (or centrally located detectors) exhibit certain time-invariance requirements that are “relaxed” in the sense that it is not strictly required that each of them remains absolutely fixed during the monitoring session. More particularly, it only needs to be assumed that the ratio of the coupling efficiencies of the centrally located sources (or centrally located detectors) remains constant during the monitoring session, and that the difference between phase errors for the centrally located sources (or centrally located detectors) remains constant during the monitoring session. These time-invariance criteria are more relaxed than a “strict” time-invariance criteria in which all coupling efficiencies and phase errors of all sources and detectors must remain fixed during the monitoring session. Notably, because the centrally located sources (or centrally located detectors) are physically nearby to each other and nestled well within the interior confines of the ATF patch, it is believed particularly realistic that the ratio of their coupling efficiencies, if not the actual values of their coupling efficiencies, will tend to remain constant throughout the monitoring session. More generally stated, one or more of the preferred embodiments described further herein is advantageously applied when it can be assumed that the particular biological volume under study has a characteristic at the beginning of the monitoring period (which can be termed a calibration period) in which both of the localized subregions (or “N” subregions if there are more than two subregions being monitored) can be considered to have a generally uniform value for the optical property to be monitored.

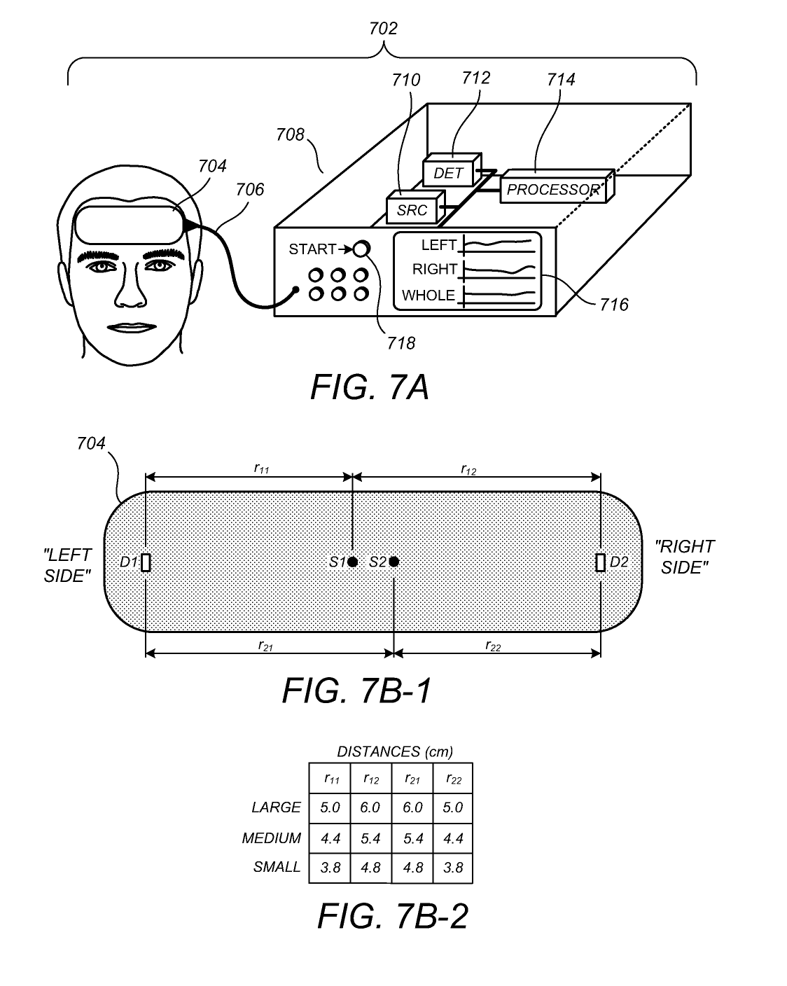

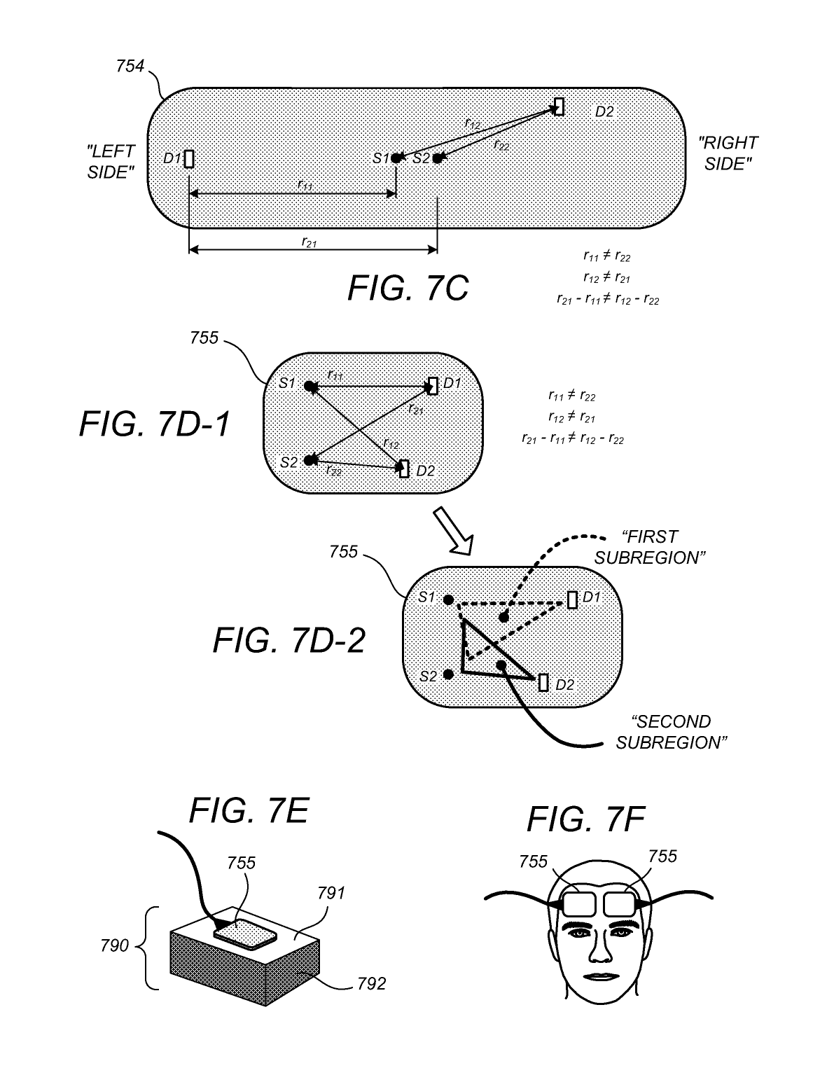

FIG. 7A illustrates an NIR cerebral oximeter 702 according to a preferred embodiment, comprising an across-the-forehead (ATF) probe patch 704 coupled via optical, electro-optical, or electrical cables 706 to a console unit 708. Console unit 708 comprises one or more optical sources 710 and optical detectors 712, each of which may be fully optical, electro-optical, or fully electrical in nature depending on the nature of the sources and detectors on the probe patch 704. For one preferred embodiment, the optical sources 710 comprise one or more laser sources, the optical detectors 712 comprise one or more photomultiplier tubes (PMTs), and the probe patch 704 consists of passive optical sources and detectors and has a general overall construction similar to one or more of the NIR probe patches disclosed in the commonly assigned and U.S. Ser. No. 12/483,610 filed Jun. 12, 2009 with the dimensions, source locations, and detector locations being as set forth herein. Console unit 708 further comprises a processor 714 coupled to control and receive information from the optical sources 710 and optical detectors 712, the processor 714 being configured, dimensioned, and programmed to achieve the functionalities described herein. Console unit 708 further comprises an output display 716 coupled to the processor 714 that simultaneously displays left, right, and whole-brain SO2 readings (and, optionally, intermediate values such as slopes, absorption coefficients, and scattering coefficients) in any of a variety of numerical and/or graphical formats. Among a variety of other control inputs, the console unit 708 further comprises a “start” button 718 that allows for user initiation of the SO2 monitoring session. The “start” button 718 can alternatively be termed a calibration button, as it instantiates a calibration process in which particular algorithm compensations (and/or other parameters) are determined based on a presumption that the optical property to be monitored is spatially homogenous throughout the different subregions of monitored tissue at that “start” time or calibration time.

FIGS. 7B-1 and 7B-2 illustrate a simplified version of the probe patch 704 and dimensions relevant thereto according to one preferred embodiment, the probe patch 704 having only two sources S1-S2 and two detectors D1-D2 positioned as shown. Different ATF probe patches having different source-detector separation distances can be provided for differently size foreheads. In other preferred embodiments there are additional sets of detectors for providing readings that are applicable for additional source-detector separation distances.

FIG. 7C illustrates a simplified version of an alternative probe patch 754 that can be used in conjunction with the NIR cerebral oximeter 702 according to a preferred embodiment. Advantageously, as will be illustrated further infra, it is not required that the prior art symmetries of FIG. 4 be present in order to achieve the desired monitoring functionalities according to the preferred embodiments, and thus the probe patch 754 is shown without those symmetries present.

FIGS. 7D-1 and 7D-2 illustrate a simplified version of an alternative probe patch 755 that can be used in conjunction with the NIR cerebral oximeter 702 according to a preferred embodiment. Whereas the non-symmetric probe patch 754 still maintains a somewhat linear configuration that defines left and right subregions (albeit non-symmetrically), analogous to that of the probe patch 704, the non-symmetric probe patch 755 represents a more quadrilateral-shaped configuration that is applicable to a more compact region of tissue. For the probe patch 755, it is required only that the sources and detectors be laid out so as to define plural subregions that are at least partially non-overlapping with each other. As illustrated in FIG. 7D-2, each partially non-overlapping subregion is defined by either a single detector with two sources of differing distances therefrom (to allow the above-described slope method to be applicable) or, alternatively, a single source with two detectors of differing distances therefrom.

FIG. 7E illustrates the probe patch 755 of FIGS. 7D-1 and 7D-2 as mounted on a surface 791 of a biological volume 790, for monitoring an optical property of the subsurface tissue 792. The biological volume 790 can generally be any part of the body, and is not limited to the head of the patient.

FIG. 7F illustrates NIR cerebral oximetry based on the probe patch 755 of FIGS. 7D-1 and 7D-2, wherein there are two probe patches 755 coupled to respective sides of the forehead of the patient. For the scenario of FIG. 7F, each probe patch 755 can provide optical property readings for two subregions (e.g., an “upper” subregion and “lower” subregion, see FIG. 7D-2) for its respective hemisphere, and/or each probe patch 755 can provide a single reading for its respective hemisphere based on an averaging or other combination of the two subregions.

In keeping with the bidirectional nature of light, for each of the preferred embodiments herein there exists a converse configuration in the form of swapped source-detector positions that is also a preferred embodiment within the scope of the present teachings and that operates in essentially the same way. For example, with reference to FIG. 7B-1, an alternative converse configuration exists in which the detectors D1 and D2 are in the center of the probe patch, and the sources S1 and S2 are at the lateral peripheries of the probe patch. The relevant mathematical formulae and functional operation of these conversely configured preferred embodiments would be readily apparent to a person skilled in the art in view of present disclosure, and need not be discussed further herein.

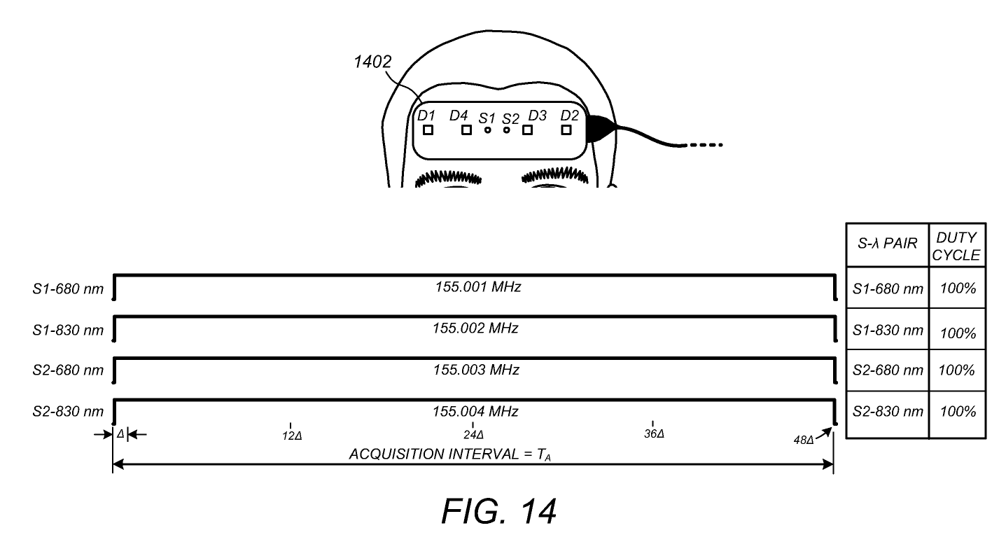

For any particular ATF patch, the operational methods and computations for the different source-detector quadruplets thereon are generally independent of each other. For example, referring briefly to the probe patch of FIG. 14, infra, measurements corresponding to the S1-S2/D1-D2 quadruplet shown in FIG. 14 can be processed to compute a first absolute SO2 value, and a separate set of measurements corresponding to the S1-S2/D3-D4 quadruplet can be processed to compute a second absolute SO2 value, with there being no dependencies between the two sets of computations. The multiple SO2 readings (and/or the multiple underlying values of the slopes, absorption coefficients, effective scattering coefficients, etc., at each wavelength) for the multiple source-detector quadruplets can be processed in any of a variety of different advantageous ways without departing from the scope of the present teachings. For example, in a two-quadruplet scenario (see FIG. 14) in which there is a “near” quadruplet (S1-S2/D3-D4) and a “far” quadruplet (S1-S2/D1-D2), the “near” readings associated with lesser penetration depths can be processed in conjunction with the “far” readings associated with deeper penetration depths to extract outputs more specific to the deep brain tissue. In one preferred embodiment, the different “near” and “far” readings are processed as described in the commonly assigned U.S. Ser. No. 12/815,696, filed Jun. 15, 2010, which is incorporated by reference herein. Because the computations for different source-detector quadruplets are substantially the same and generally independent of each other, the preferred methods for bilateral and whole-head SO2 monitoring will be detailed further herein for the simplified, single quadruplet system (S1-S2/D1-D2) of FIG. 7C.

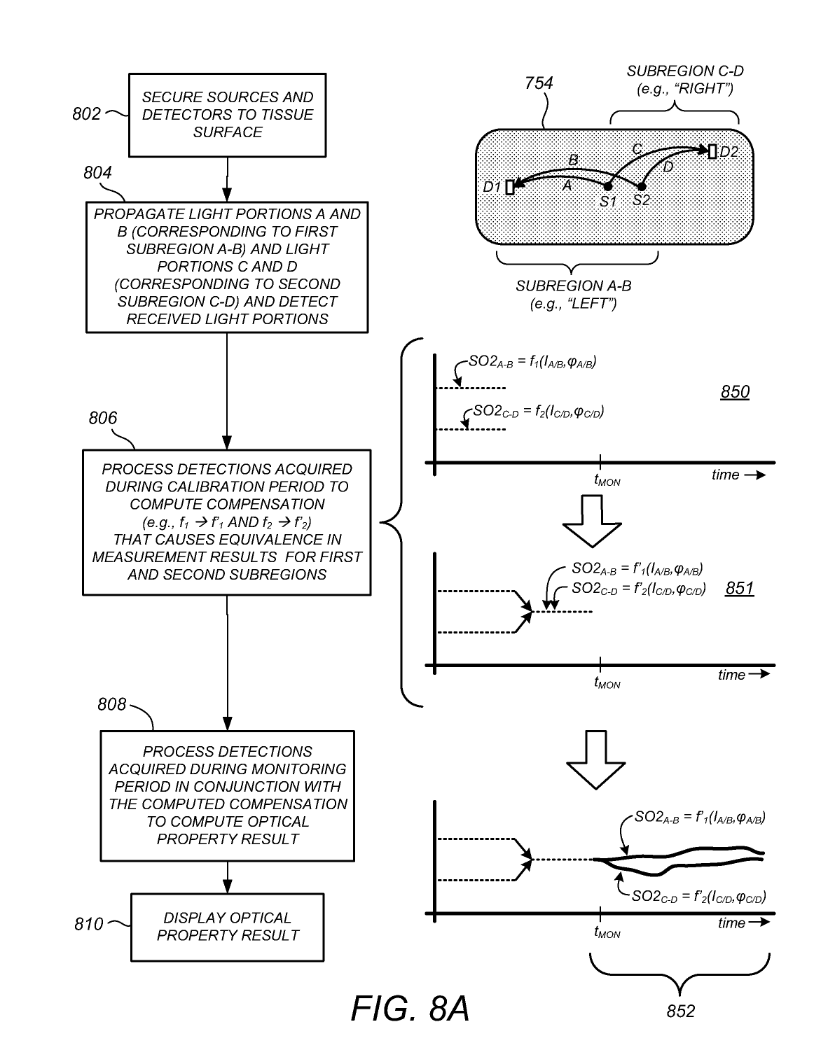

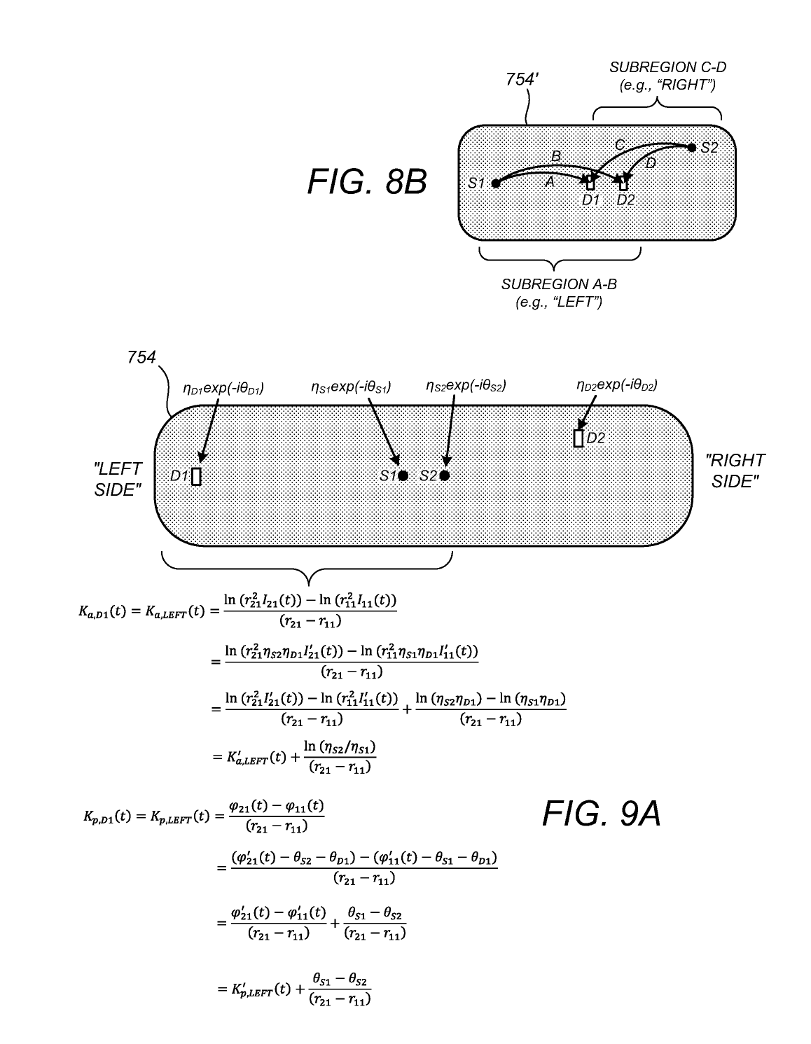

FIG. 8A illustrates near-infrared spectrophotometric (NIR) monitoring of a biological volume of a patient according to a preferred embodiment. At step 802, the NIR sources and detectors, as contained for example on the probe patch 754, are secured to a surface of the biological volume. Referring ahead briefly to FIG. 8B, in keeping with the bidirectional nature of light, there exists a converse probe patch 754′ for which the present description is equivalently applicable, in the form of swapped source-detector positions relative to the probe patch 754. Upon mounting and securing of the probe patch, a calibration interval can begin, such as by the user pressing the “start” button 718, which is followed by a monitoring interval. The calibration interval should usually last a few seconds, but can be substantially lesser or greater without departing from the scope of the present teachings. The monitoring interval can be anywhere from a few minutes to several hours, depending on the nature of the clinical procedure (e.g., during surgery, during post-operative recovery, during other patient testing, etc.) in association with which the patient monitoring may be taking place. During each of a calibration interval and the subsequent monitoring interval (step 804), a first portion of light (denoted “A” in FIG. 8A) is propagated from a first optical source S1 through the medium to the first optical detector D1, a second portion of light (“B”) is propagated from the second optical source S2 through the medium to the first optical detector D1, a third portion of light (“C”) is propagated from the first optical source S1 through the medium to the second optical detector D2, and a fourth portion of light (“D”) is propagated from the second optical through the medium to the second optical detector.

At step 806, detections of the first light portion “A”, second light portion “B”, third light portion “C”, and fourth light portion “D” that were acquired during the calibration time interval are processed to compute at least one algorithm compensation that causes (i) a first result related to the optical property based on the first and second light portions “A” and “B”, which correspond to the subregion A-B (i.e., the “left” side), to be substantially equal to (ii) a second result related to the optical property based on the third and fourth light portions “C” and “D”, which correspond to the subregion C-D (i.e., the “right” side). The first and second results to which algorithm compensation is applied can be, for example, a left-side SO2 reading and a right-side SO2 reading, respectively, computed according to the “slope” method. Alternatively, the first and second results to which algorithm compensation is applied can be intermediate values, such as the intensity-based slope factor Kₐ, for the left and right sides as would be computed on the way to computing an eventual SO2 end result. Shown by way of example in FIG. 8A is a plot 850 of the SO2 results for the left side (SO2_{A-B}) and the right side (SO2_{C-D}) as would be computed by the slope method in a direct or uncompensated form based on readings taken during the calibration interval. Then, shown in FIG. 8A in the plot 851 are the results SO2_{A-B} and SO2_{C-D} as they appear in compensated form, wherein the algorithms for computing these results have been compensated in a way that forces these values to be equal.

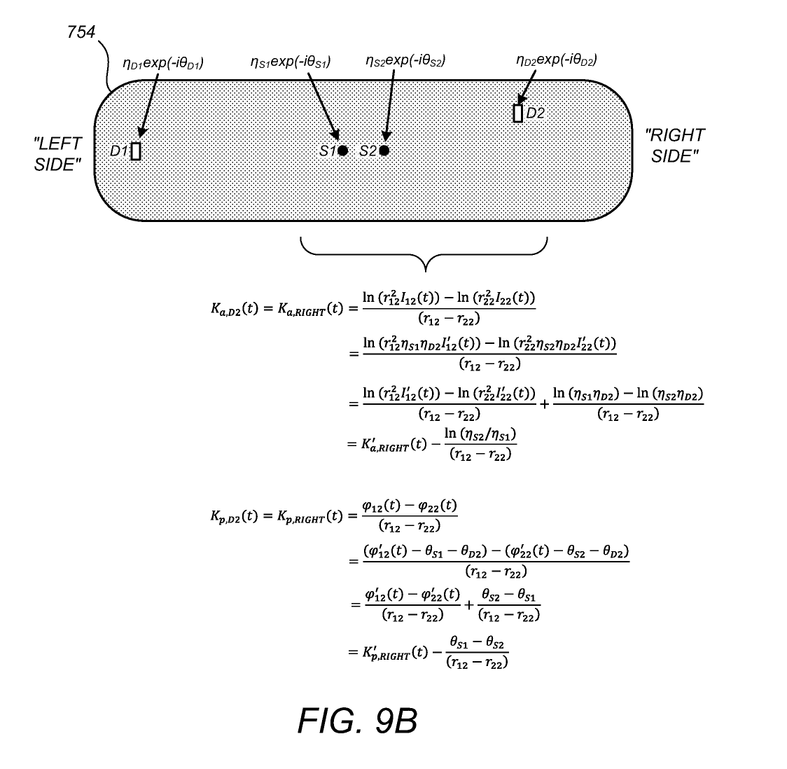

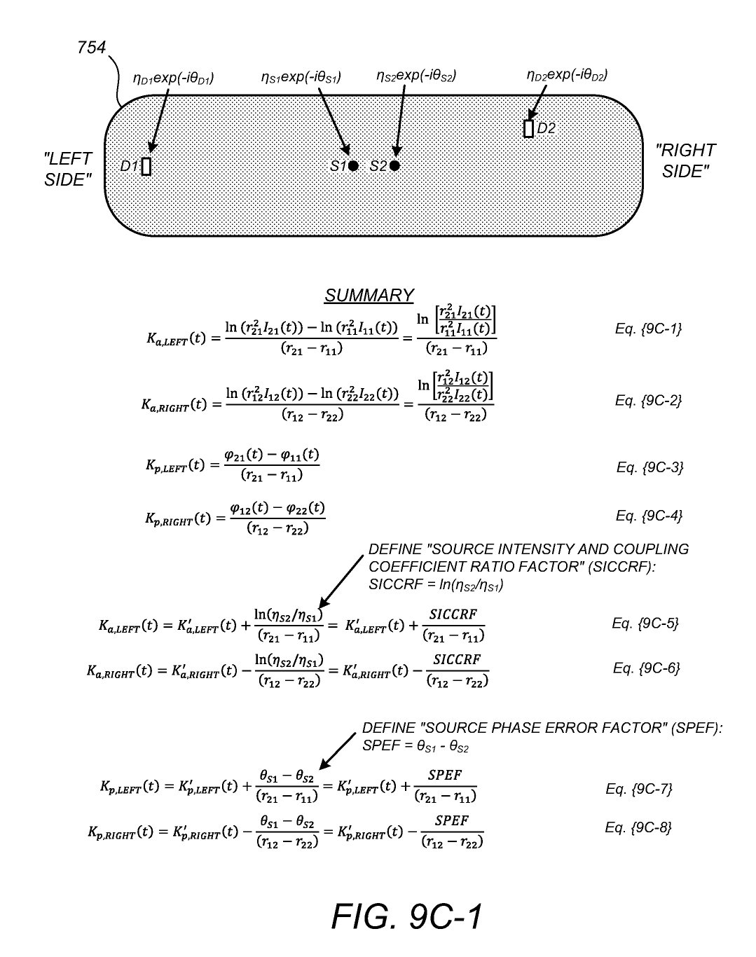

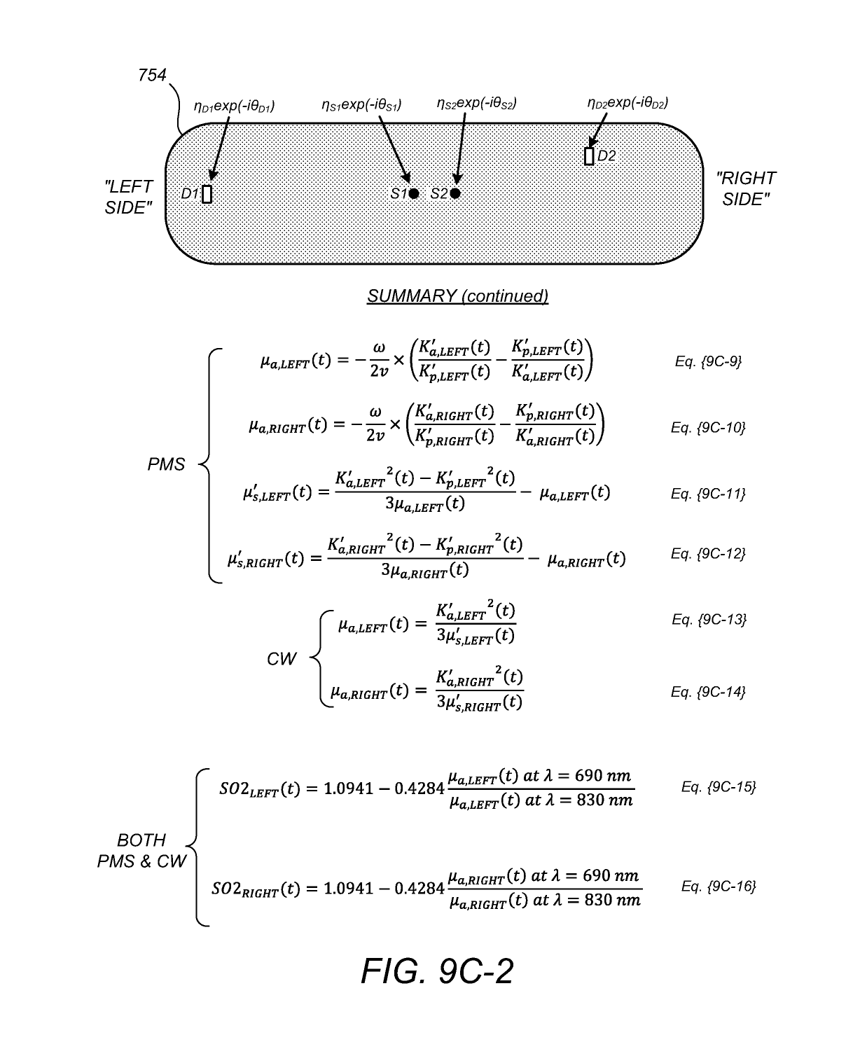

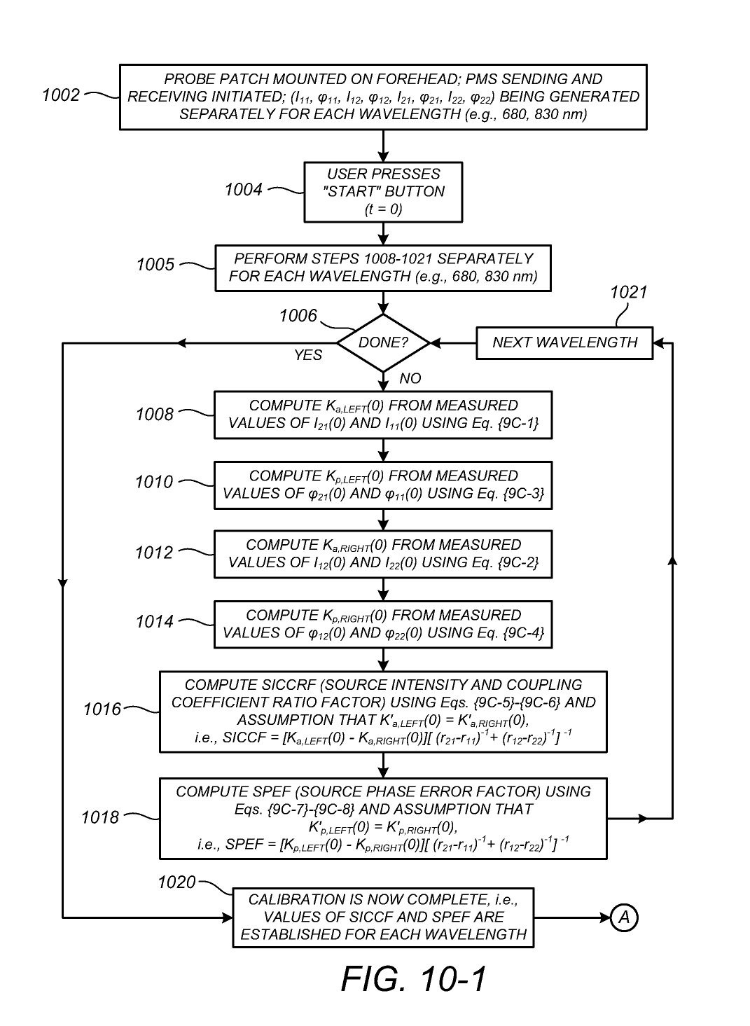

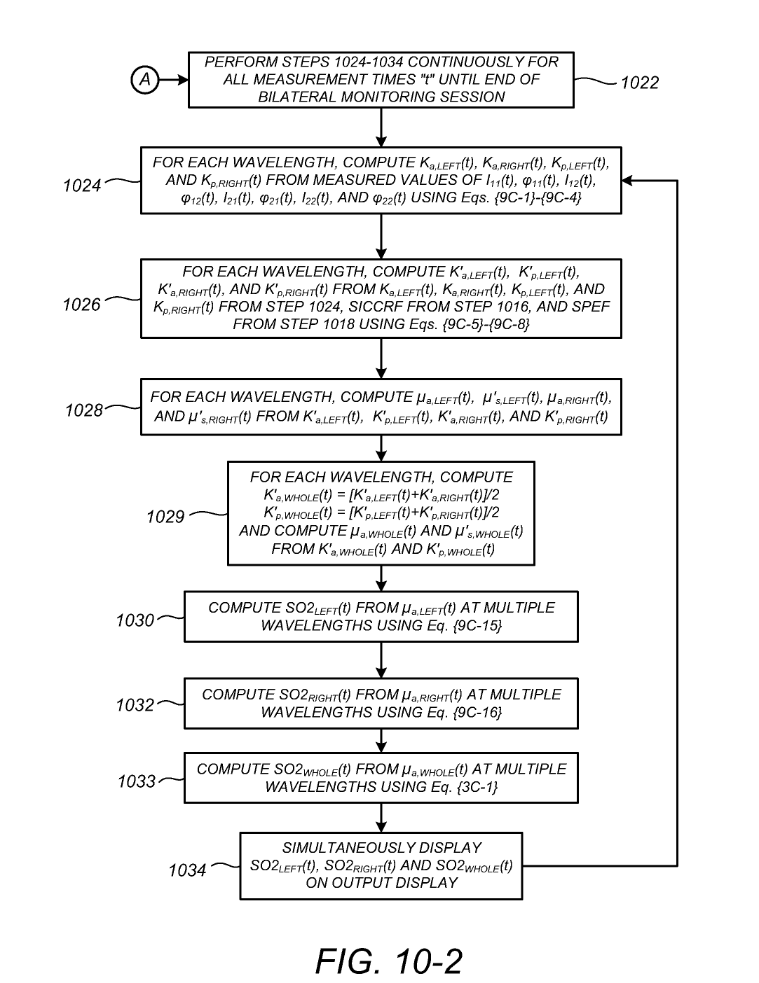

Examples of algorithm compensations applied to cause the identical results for the two respective subregions are disclosed further infra with respect to FIGS. 9A-9C and FIG. 10. For the example of FIG. 8A, the algorithm compensations are simply represented by the use of primed (′) versions of the result computation algorithms. According to one preferred embodiment, the applied algorithm compensation(s) are selected to relate to at least one non-ideality associated with one or more of the intensity of the optical sources, the sensitivity of the optical detectors, the coupling efficiency of light from the optical sources into the medium, and the coupling efficiency of light from the medium to the optical detectors. For example, one or more correction factors can be applied to change the values of the source intensity/coupling coefficients, detector efficiency/coupling coefficients, and/or phase error coefficients (for PMS implementations) used on the slope method equations such that the results of the slope method equations yield the same result for the two different subregions. Stated differently, the calibration process for a multi-subregion monitoring system according to a preferred embodiment harnesses a presumption that the optical property itself is spatially homogenous throughout the multiple subregions during the calibration interval, and that any differences between readings taken during that calibration interval are attributable to determinable non-idealities in the measurement system. The readings taken during the calibration interval are then used to determine the extent of those non-idealities and to compensate for them during the remainder of the monitoring session. Subsequently, if the multiple localized readings begin to depart from each other during the monitoring interval, those differences are indeed attributed to actual biological fluctuations in the patient (e.g., an ischemic condition in the left or right side of the brain), under a presumption that the non-idealities (or at least particular ratios related to those non-idealities, as described further infra) have remained constant during the post-calibration monitoring interval.

At step 808, subsequent to the calibration process of step 806, detections of the light portions “A” through “D” proceed throughout the monitoring interval, and the optical property is computed using the detected light in conjunction with the one or more compensation factors computed at step 806. At step 810, the resultant optical property is displayed on an output display, as illustrated by the plots 852 showing the SO2 level for the left (A-B) and right (C-D) sides of the brain, respectively. Notably, as described above in relation to step 806, it is not required that the ultimate result (in this case, SO2) be computed for each of the different subregions in determining the algorithm compensations during the calibration phase. Rather, it can be an intermediate result that is computed for each subregion (such as a slope factor), or some other property for each subregion for which homogeneity among subregions would be implicated under an assumption that the ultimate property to be measured is known to be homogeneous throughout the subregions.

For one preferred embodiment, during each of the calibration interval and monitoring interval, each of the light portions “A” through “D” comprises a combination of light portions corresponding to two (or more) different wavelengths (e.g., 680 nm and 830 nm), wherein only a single source is emitting at any particular instant in time, and that emitting source is emitting only a single wavelength at any particular instant in time. The different sources and wavelengths are individually cycled through on a repeated basis through successive periods that are termed herein acquisition intervals. By way of example, for an exemplary acquisition interval of one second, the following sequence may be carried: S1 emitting at 680 nm for 0.25 seconds to provide light portions A(680) and C(680), followed by S1 emitting at 830 nm for 0.25 seconds to provide light portions A(830) and C(830), followed by S2 emitting at 680 nm for 0.25 seconds to provide light portions B(680) and D(680), followed by S2 emitting at 830 nm for 0.25 seconds to provide light portions B(830) and D(830). The process then repeats every second throughout the calibration and monitoring intervals. Any particular light portion at any particular wavelength thereby only has an active duty cycle of 25% (0.25 seconds out of every second).

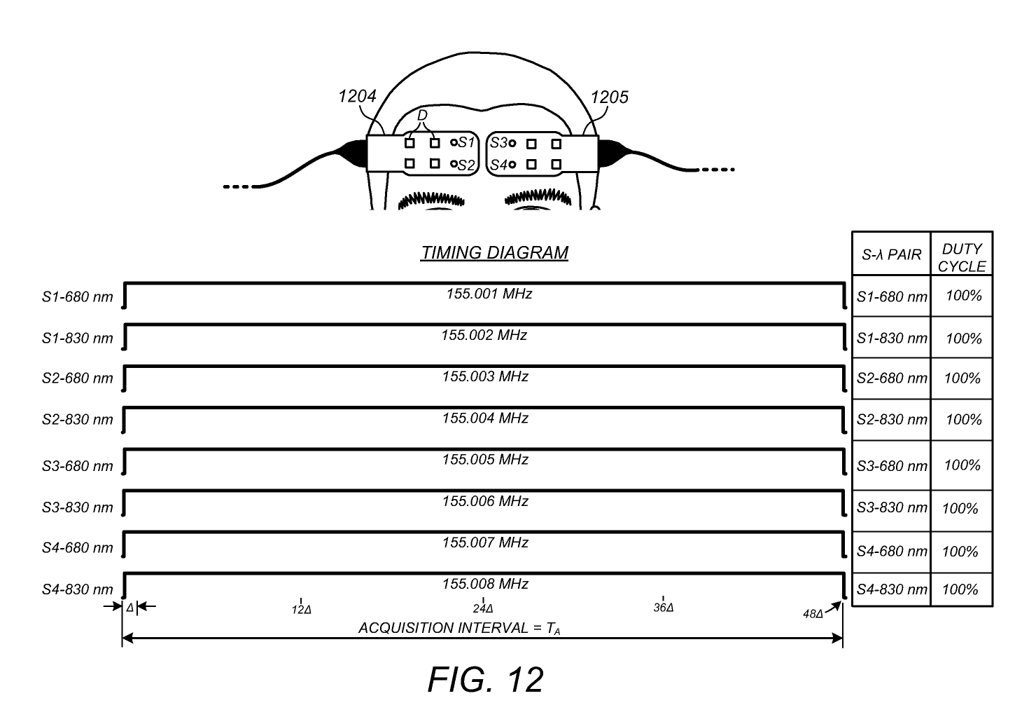

For another preferred embodiment similar to one or more preferred embodiments detailed further hereinbelow in relation to FIGS. 12-17, each of the light portions “A” through “D” comprises a combination of light portions corresponding to two (or more) different wavelengths (e.g., 680 nm and 830 nm), wherein both sources are emitting simultaneously and continuously at both wavelengths, and wherein a frequency division multiplexing scheme is used so that the detectors can individually detect each distinct light portion at each distinct wavelength. By way of example, source S1 may be continuously emitting at 680 nm at a modulation frequency of 155.001 MHz, source S1 may be continuously emitting at 830 nm at a modulation frequency of 155.002 MHz, source S2 may be continuously emitting at 680 nm at a modulation frequency of 155.003 MHz, and source S2 may be continuously emitting at 830 nm at a modulation frequency of 155.004 MHz. Each of the detectors D1 and D2 receives all signals simultaneously and separates (demultiplexes) them from each other based on their distinct modulation frequencies. Any particular light portion at any particular wavelength thereby has an active duty cycle of 100% which, as described further hereinbelow, can provide for enhanced signal to noise performance as compared to scenarios in which the there is a lesser duty cycle.

FIGS. 9A-9C and FIG. 10 illustrate a particular application of the general method of FIG. 8A, in the context of a PMS-based spectrophotometry system using the probe patch 754 based on two representative wavelengths of 680 nm and 830 nm. FIGS. 9A-9C illustrate equations for adapting the slope method of absorption coefficient and effective scattering coefficient computation to a bilateral NIR cerebral oxygenation monitor using a reduced-element across-the-forehead (ATF) patch according to a preferred embodiment. FIG. 9A illustrates equations that represent the measured slopes Kₐ and Kₚ as “seen” by the left side detector D1 for the distance interval r₁₁ to r₂₁, which are denoted K_{a,LEFT}(t) and K_{p,LEFT}(t), respectively. The left-side measured slope K_{a,LEFT}(t) is computed from the measured light intensity values I₂₁(t) and I₁₁(t) as shown, while the measured left-side phase slope K_{p,LEFT}(t) is computed from the measured phase values φ₂₁(t) and φ₁₁(t) as shown. FIG. 9B illustrates equivalent equations applicable for the right side detector D2.

As illustrated in FIG. 9C, which collects and compares the slope equations from FIGS. 9A-9B, the measured left-side slope K_{a,LEFT}(t) differs from the ideal left-side slope K′_{a,LEFT}(t) only by the log of the ratio of the coupling efficiencies of the centrally located sources S1 and S2, termed herein a source intensity and coupling coefficient ratio factor (SICCRF), divided by the known quantity r₂₁-r₁₁ {Eq. 9C-5}. The measured right-side slope K_{a,RIGHT}(t) differs from the ideal right-side slope K′_{a,RIGHT}(t) only by the SICCRF (oppositely signed), divided by the known quantity r₁₂-r₂₂ {Eq. 9C-6}. Moreover, the measured left-side phase slope K_{p,LEFT}(t) differs from the ideal left-side phase slope K′_{p,LEFT}(t) only by the difference of the phase errors of the centrally located sources S1 and S2, termed herein a source phase error factor (SPEF), divided by the known quantity r₂₁-r₁₁ {Eq. 9C-7}. The measured right-side phase slope K_{p,RIGHT}(t) differs from the ideal right-side phase slope K′_{p,RIGHT}(t) only by the SPEF (oppositely signed), divided by the known quantity r₁₂-r₂₂ {Eq. 9C-8}. According to a preferred embodiment, these relationships are uniquely combined with the bilaterality assumptions set forth above (including homogeneity at time 0) to permit the separate computation of K′_{a,LEFT}(t), K′_{a,RIGHT}(t), K′_{p,LEFT}(t), and K′_{p,RIGHT}(t) throughout the monitoring session, which are then used to compute separate, absolute left-side (SO2_{LEFT}(t)) and right-ride (SO2_{RIGHT}(t)) oxygen saturation values throughout the monitoring session. Briefly stated, when the user presses the “Start” button at the beginning (t=0) of the monitoring session, the algorithm compensation referenced at step 806 of FIG. 8A proceeds by a determination of the values for SICCRF and SPEF (calibrated) for each NIR radiation wavelength based on (i) measured intensity and phase values at t=0, and (ii) the assumption that K′_{a,LEFT}(0)=K′_{a,RIGHT}(0) and K′_{p,LEFT}(0)=K′_{p,RIGHT}(0). Then, for all times t>0 after the calibration is complete, the values of K′_{a,LEFT}(t), K′_{a,RIGHT}(t), K′_{p,LEFT}(t), and K′_{p,RIGHT}(t) are computed based on (i) the measured intensity and phase values at time “t”, and (ii) the determined (calibrated) values of SICCRF and SPEF.

Stated somewhat more broadly, operation of a bilateral NIR cerebral oximeter using a reduced-element ATF patch according to one preferred embodiment is based on a modified version of the slope method in which left-side slopes and right-side slopes are individually computed, wherein (i) at the quiescent beginning of the monitoring session, it is presumed that any differences in the left-side slopes versus the right-side slopes are attributable to coupling efficiency and/or phase error differences among the sources and detectors because the SO2 distribution is assumed uniform across both left and right hemispheres, and (ii) during the subsequent course of the monitoring session, it is presumed that any change in the left-side slopes or right-side slopes is attributable to timewise physical changes in the SO2 values in that hemispheres because the coupling efficiency and/or phase error differences are presumed to be fixed in time.

Notably, for the converse preferred embodiment in which the detectors D1-D2 are centrally located and the sources S1-S2 are at the left and right ends, it can be shown that the equations turn out similarly to FIG. 9C except that the source intensity and coupling coefficient ratio factor (SICCRF) becomes a detector sensitivity and coupling coefficient ratio factor (DSCCRF) equal to the log of the ratio of the coupling efficiencies of the centrally located detectors D1 and D2, and the source phase error factor (SPEF) becomes a detector phase error factor (DPEF) equal to the difference of the phase errors of the centrally located detectors D1 and D2. Thus, in the a more general expression of the preferred embodiments, the SICCRF could be replaced in the present description by a factor termed the centrally located element coupling coefficient ratio factor (CLECCRF) and the SPEF could be replaced in the present description by a factor termed the centrally located element phase error factor (CLEPEF).

FIG. 10 illustrates bilateral NIR cerebral oxygenation level monitoring according to a preferred embodiment. As the process begins at step 1002, the ATF patch has been mounted and the system has begun to acquire intensity and phase measurements during a calibration interval (the time is arbitrarily set to “0” for the time at which calibration, i.e., algorithm compensation, takes place). A set of quiescent readings for the measured intensities and measured phases is established and maintained at this time, based for example on a running 10-second averaging interval (or other suitable averaging interval) to ensure a set of smooth and reliable intensity and phase values at t=0 when the calibration process will begin. Then, with the patient in a quiescent state such that the bilateral assumptions supra are valid (e.g. the surgery operation has not yet begun and the ATF patch is safely secured to the forehead), the user presses the start button (step 1004) at time t=0 to start the calibration process, which is carried out separately for each wavelength. At steps 1008-1014, the measured slopes K_{a,LEFT}(0), K_{a,RIGHT}(0), K_{p,LEFT}(0), and K_{p,RIGHT}(0) are computed from the quiescent measured intensities and phases I₁₁(0), φ₁₁(0), I₁₂(0), φ₁₂(0), I₂₁(0), φ₂₁(0), I₂₂(0), and φ₂₂(0). At steps 1016-1018, the SICCRF and SPEF are computed based on (i) the measured slopes K_{a,LEFT}(0), K_{a,RIGHT}(0), K_{p,LEFT}(0), and K_{p,RIGHT}(0), and (ii) the assumptions that K′_{a,LEFT}(0)=K′_{a,RIGHT}(0) and K′_{p,LEFT}(0)=K′_{p,RIGHT}(0). The calibration process for that wavelength is then complete (step 1020), and the process is repeated for each wavelength such that separate values of SICCRF and SPEF are established for each wavelength.

Subsequent to the calibration process, for all times t>0 (it can be assumed for purposes of this description that the calibration process took a negligible amount of time immediately after t=0), the known (calibrated) values of SICCRF and SPEF are used in conjunction with the ongoing measured slope values to compute the ideal slope values for the left side, right side, and whole-brain for each wavelength, which are then used as the basis for the left side, right side, and whole-brain SO2 values. Thus, at step 1024, the measured slope values K_{a,LEFT}(t), K_{a,RIGHT}(t), K_{p,LEFT}(t), and K_{p,RIGHT}(t) are computed from the measured intensities and phases at time “t”. At step 1026, the ideal slope values K′_{a,LEFT}(t), K′_{a,RIGHT}(t), K′_{p,LEFT}(t), and K′_{p,RIGHT}(t) are computed based on K_{a,LEFT}(t), K_{a,RIGHT}(t), K_{p,LEFT}(t), and K_{p,RIGHT}(t) and the values of SICCRF and SPEF. At step 1028, the absorption coefficients and effective scattering coefficients are computed from K′_{a,LEFT}(t), K′_{a,RIGHT}(t), K′_{p,LEFT}(t), and K′_{p,RIGHT}(t). For whole-brain monitoring, the value of K′_{a,WHOLE}(t) is computed as the average of K′_{a,LEFT}(t) and K′_{a,RIGHT}(t), the value of K′_{p,WHOLE}(t) is computed as the average of K′_{p,LEFT}(t) and K′_{p,RIGHT}(t), and the corresponding absorption coefficients and effective scattering coefficients are computed therefrom at step 1029. Finally, at steps 1030-1033 the values of SO2_{LEFT}(t), SO2_{RIGHT}(t), and SO2_{WHOLE}(t) are computed from the absorption coefficients at the multiple wavelengths, and at step 1034 they are displayed on the output display 716.

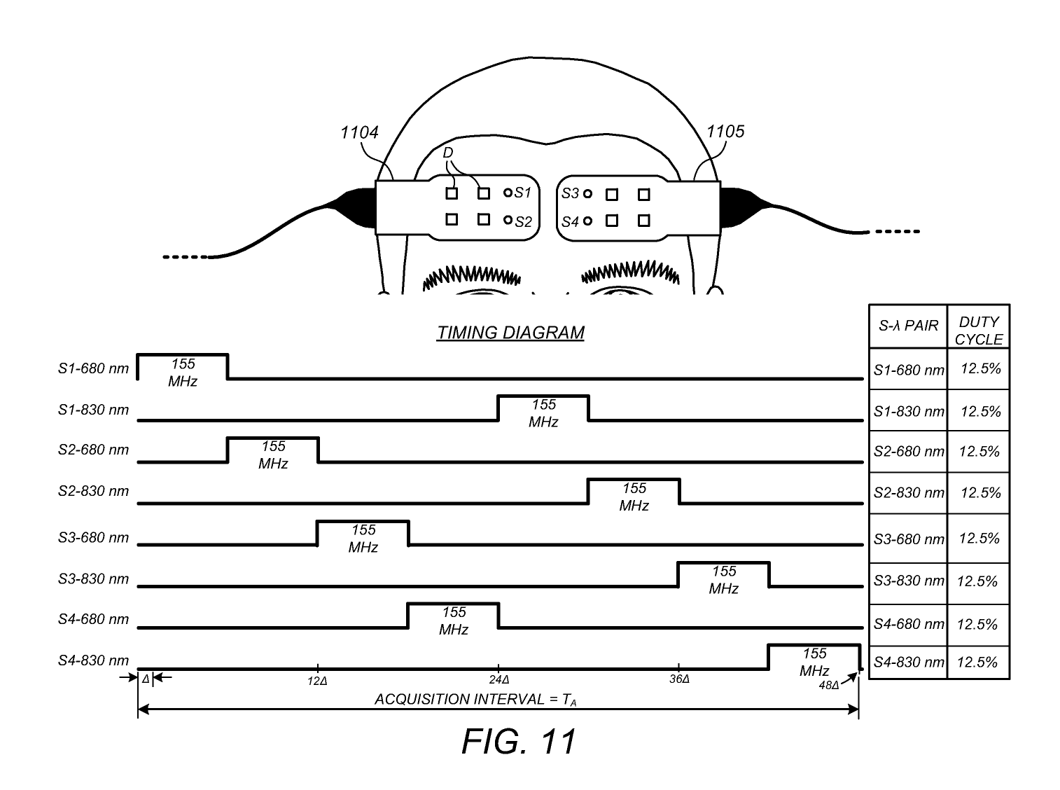

FIG. 11 illustrates NIR probe patches 1104 and 1105 mounted on the forehead of a patient and a corresponding source timing diagram corresponding to a scenario in which there is a relatively low duty cycle for any particular optical signal at any particular wavelength. Probe patch 1104 includes two source ports S1 and S2 and four detector ports D. The probe patch 1105 also includes two source ports S3 and S4 and four detector ports. For the example of FIG. 11, it is presumed that a PMS (phase modulated spectrophotometry) scheme is used in which there are two NIR wavelengths (680 nm and 830 nm) and a modulation frequency of 155 MHz. During each acquisition cycle T_{A}, which is typically on the order of 1 second, there needs to be provided individually measured amplitudes and phases for each of the individual wavelengths 680 nm and 830 nm for each individual source port/detector port pair on each of the NIR probe patches 1104 and 1105. According to the example of FIG. 11, this is achieved by firing each source port/wavelength pair during a distinct time interval that does not overlap with any other source port/wavelength pair. Thus, each of the following source port/wavelength pairs emits during a distinct time interval: S1—680 nm; S1—830 nm; S2—680 nm; S2—830 nm; S3—680 nm; S3—830 nm; S4—680 nm; and S4—830 nm. Each detector actively detects (“listens”) whenever any of the source ports on that same NIR probe patch are firing.

By firing each source port/wavelength pair during a distinct time interval, it is ensured that each detector port achieves a clear, individualized “channel” with each source port/wavelength pair (i.e., with each individual wavelength emitted at each individual source port), without interference or stray radiation from other sources or other wavelengths. As used herein, “duty cycle” refers to the percentage of time that any particular “channel” (i.e., any particular source port/detector port/wavelength triplet) is actively providing measured amplitudes and phases during the tissue monitoring session. It can be readily seen that all detector ports will have the same duty cycle for any particular source port/wavelength pair, because the detector ports can operate (“listen”) independently of each other. Accordingly, unless indicated otherwise, duty cycles are presented herein only in terms of the particular source port/wavelength pair (e.g., the duty cycle for S1—680 nm, the duty cycle for S1—830 nm, etc.), with it being understood that such duty cycle applies across all of the different detectors on the NIR probe patch.

For the example of FIG. 11, assuming that all source port/wavelength pairs are given equal treatment, the maximum achievable duty cycle is 12.5% for each source port/wavelength pair. More generally, for systems having “N” different source ports and “M” different wavelengths, the maximum achievable duty cycle is 1/(NM). Most prior art cerebral oximetry systems exhibit duty cycles that are well below the maximum achievable duty cycle due to various hardware considerations, such as detection “setup time” for synchronizing to the next active source port/wavelength pair. Some known prior art cerebral oximetry systems exhibit duty cycles that are even as low as 1% for each source port/wavelength pair.

FIG. 12 illustrates NIR probe patches 1204 and 1205 mounted on the forehead of a patient and a corresponding source timing diagram for an NIR cerebral oximetry system according to a preferred embodiment, wherein each source port/wavelength pair emits at a different modulation frequency, and wherein an overall received signal at each detector port is processed to separately extract therefrom a plurality of individual received signals based on their different modulation frequencies, each individual received signal corresponding to a respective one of the source port/wavelength pairs. This provides the ability for multiple source port/wavelength pairs to be emitting simultaneously, because each detector is able to distinguish each individual “channel” based on its modulation frequency. For one preferred embodiment, for a system having “N” different source ports and “M” different wavelengths, all “NM” source port/wavelength pairs emit simultaneously and continuously throughout the monitoring session, each having a different modulation frequency, thereby providing 100% duty cycle (“full duty cycle”).

By providing full duty cycle for each individual source port/detector port/wavelength triplet (“channel”) in the preferred embodiment of FIG. 12, as compared to a duty cycle of 1/(NM) for each such channel in the example of FIG. 11, there is provided a factor of NM more data points over any particular sampling period for each channel. This, in turn, provides for an increase in the signal to noise ratio (SNR) for each channel. It can be shown that the improvement in signal to noise performance for each channel can be estimated by the square root of the factor by which the number of data points per sampling interval has increased. Thus, where the number of data points per sampling interval has increased by a factor of NM, the improvement in signal to noise performance is roughly the square root of NM. For the preferred embodiment of FIG. 12, where the number of source ports “N” is 4, the number of wavelengths “M” is 2, and the number “NM” of source port/wavelength pairs is 8, the improvement in signal to noise performance over the example of FIG. 11 is roughly 280%. Another advantage is that, since the monitoring is continuous, there are no inefficiencies caused by the need for repeated “setup times” during each acquisition interval, as is the case for the example of FIG. 11.

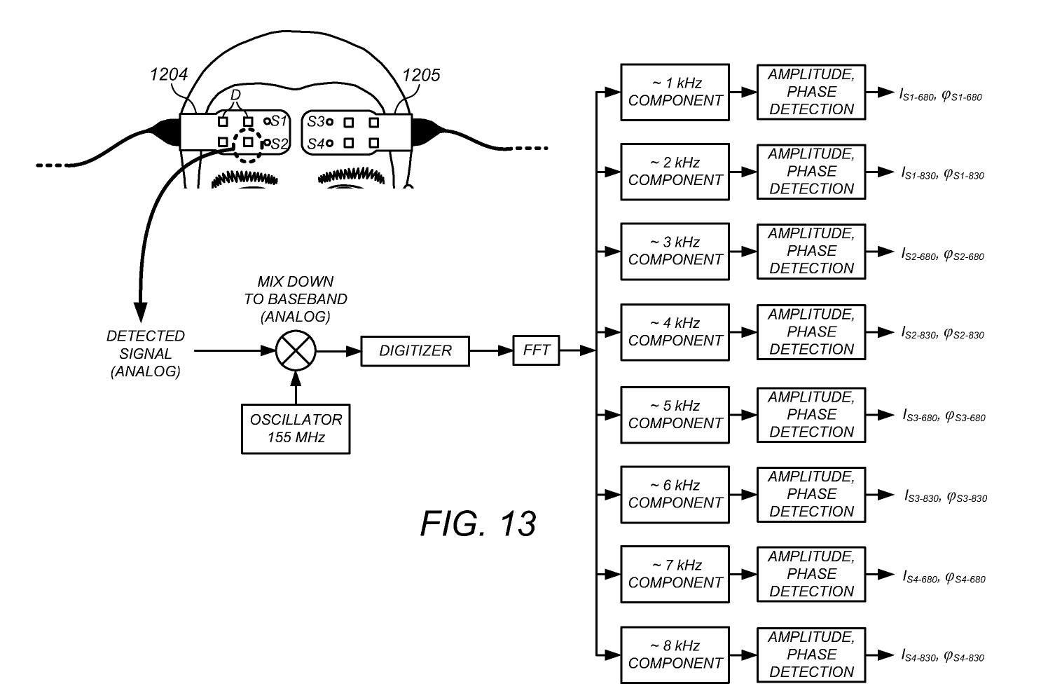

FIG. 13 illustrates a block diagram of signal processing circuitry associated with NIRS monitoring according to a preferred embodiment. The block diagram of FIG. 13 is individually applicable for each distinct detector port (detector). The overall received signal at a particular detector port, which is in analog electrical form (e.g., as the output of a photomultiplier tube or semiconductor photodiode), is processed to separately extract therefrom a plurality of individual received signals (amplitudes I and phases φ) based on their different modulation frequencies. As illustrated, each individual received signal (e.g., I_{S1-680}, φ_{S1-680}) corresponds to a respective one of the source port/wavelength pairs (e.g., S1—680 nm) with which that detector port establishes NM/2 corresponding respective source port/detector port/wavelength triplets (“channels”). For the more general case in which all sources and detectors are on the same probe patch, the number of source port/detector port/wavelength triplets (“channels”) established for each detector port is NM. For one preferred embodiment, the circuit of FIG. 13 is replicated for each different detector port in the NIRS monitoring system.

It is to be appreciated that the particular modulation frequencies, channel spacings, etc. that are set forth FIGS. 12-13 are presented by way of example only, and not by way of limitation, although for one preferred embodiment, it has been found advantageous from a hardware and signal processing perspective to use channel spacings (here, 1 kHz) that are relatively low compared to the “base” modulation frequency (here, 155 MHz) so that only one analog mixer is needed and so that the baseband digital processing (FFT, channel filtering) can be implemented with relatively inexpensive hardware. More generally, for one preferred embodiment it has been found useful in PMS-based systems to have the different modulation frequencies each be greater than 100 MHz and yet differ from each other by less than 100 kHz. For another preferred embodiment it has been found useful in PMS-based systems to have the different modulation frequencies each be greater than 100 MHz and yet differ from each other by less than 1 MHz.

FIG. 14 illustrates an across-the-forehead (ATF) NIR probe patch 1402 mounted on the forehead of a patient and a corresponding source timing diagram for an NIR cerebral oximetry system according to a preferred embodiment. The ATF patch 1402 is preferably similar to that described in Ser. No. 12/826,218, supra, which is incorporated by reference herein, the ATF patch 1402 being particularly advantageous in providing bilateral monitoring of cerebral oxygenation levels using a reduced number of source/detector elements and/or in providing localized optical property readings without requiring certain source-detector symmetries. It has been found particularly advantageous to use the full-duty cycle methods of FIGS. 12-13, supra, in combination with the teachings of Ser. No. 12/826,218, supra, at least due to the large source-detector separation distances S1-D2 and S2-D1 that can be realized, which can be up to 6 cm or even greater, and whose operation can benefit greatly from the improved signal to noise performance provided by, according to a preferred embodiment, having the source port/wavelength pairs (S1—680 nm, S1—830 nm, S2—680 nm, and S2—830 nm) all emitting simultaneously at different modulation frequencies (155.001 MHz, 155.002 MHz, 155.003 MHz, and 155.004 MHz, respectively), and processing the overall received signal at each detector port to individually extract therefrom the received signals corresponding to each respective source port/wavelength pair.

Provided in conjunction with each of the preferred embodiments is a console unit coupled via optical, electro-optical, or electrical cables to the NIR probe patch and comprising one or more optical sources and optical detectors, each of which may be fully optical, electro-optical, or fully electrical in nature depending on the nature of the sources and detectors on the NIR probe patch. For one preferred embodiment, the optical sources comprise one or more laser sources, the optical detectors comprise one or more photomultiplier tubes (PMTs), and the NIR probe patch consists of passive optical sources and detectors and has a general overall construction similar to one or more of the NIR probe patches disclosed in the commonly assigned U.S. Ser. No. 12/483,610 filed Jun. 12, 2009, which is incorporated by reference herein, except that the dimensions, source locations, and detector locations are as set forth herein and/or in Ser. No. 12/826,218, supra. The console unit further comprises a processor and analog/digital hardware coupled to control and receive information from the optical sources and optical detectors, the processor and analog/digital hardware being configured, dimensioned, and programmed to achieve the functionalities described herein. The console unit further comprises an output display coupled to the processor that displays the SO2 readings in real time.

Each of the source ports on the NIR probe patch can be optically coupled to the optical sources of the console unit so as to simultaneously emit optical signals at each of the different wavelengths (e.g., 680 nm and 830 nm) being used. Alternatively, each source port can be spatially divided into multiple sub-ports, each sub-port simultaneously emitting at a different wavelength (for example, the source port/wavelength pair S1—680 nm being provided at a first sub-port of source port S1 and the source port/wavelength pair S1—830 nm being provided at a second sub-port of source port S1).

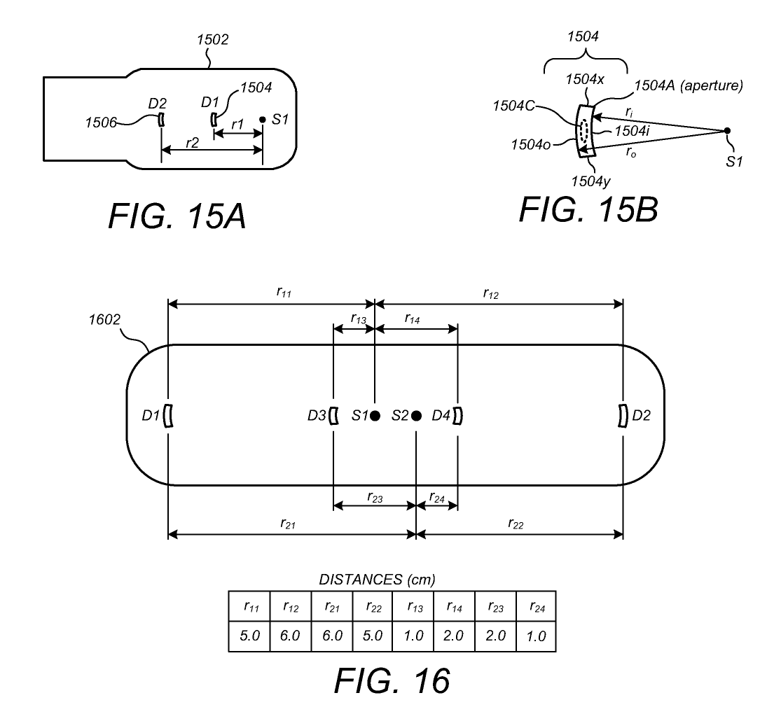

FIGS. 15A-15B illustrate an NIR probe patch 1502 having curved-edge detection apertures according to a preferred embodiment. The NIR probe patch 1502 is wearable on a surface of the tissue volume of the patient and comprises a source S1, a first detector D1, and a second detector D2. The probe patch 1502 is configured to maintain the source S1, the first detector D1, and the second detector D2 in secured contact with the surface of the tissue volume throughout the patient monitoring session. The detector D1 includes an aperture 1504 formed in the tissue-facing surface of the probe patch 1502, wherein light must travel through the aperture 1504 in order to be detected. The detector D2 likewise includes an aperture 1506. With reference to the more detailed drawing of the aperture 1504 in FIG. 15B, the aperture comprises a central area 1504C, a nearer edge 1504i that is nearer to the source S1 than the central area 1504C, and a farther edge 1504o that is farther from the source S1 than the central area 1504C. The aperture 1504 further comprises side edges 1504x and 1504y. According to a preferred embodiment, the nearer edge 1504i and farther edge 1504o are each curved inward toward the location of the source S1 (which can also be termed a source port location). In one preferred embodiment, the side edges 504x and 504y each correspond to a radial line extending through the source port location. In one preferred embodiment, the aperture 1504 has an arcuate slit-like character in that it is relatively narrow in a depthwise direction from the source S1 (i.e., in the direction of a radial line extending from the source S1) and relatively long in a tangential direction (i.e., in the direction of a tangent to a radial line extending from the source S1). For the example of FIG. 15B, the aperture 1504 is roughly three times as long in the tangential direction than in the depthwise direction. In other preferred embodiments, the aperture 1504 is at least five times as long in the tangential direction than in the depthwise direction, making its overall shape even more slit-like.

In one preferred embodiment, the nearer edge 1504i has a generally constant radius of curvature rᵢ and the farther edge 1504o has a generally constant radius of curvature rₒ, wherein each of the curvatures rᵢ and rₒ is equal to an average distance r1 of the aperture 1504 from the source S1. Likewise, the nearer and farther edges of aperture 1506 each have a curvature radius equal to an average distance r2 of the aperture 1506 from the source S1. In another preferred embodiment, the curvatures rᵢ and rₒ of aperture 1504 are equal to 0.5 times r1, or are equal to another fixed percentage of r1, which can be empirically tuned.

FIG. 16 illustrates the use of curved apertures similar to those of FIGS. 15A-15B in conjunction with an across-the-forehead (ATF) NIR probe patch 1602 that is otherwise similar to the ATF patch 1402 of FIG. 14, supra. For one preferred embodiment, the radius of curvature of each of the nearer and farther edges of each detection port (detection aperture) is the average of the distances to the different source ports S1 and S2. Thus, for example, the radius of curvature of each of the nearer and farther edges of detection aperture D3 is equal to (r₁₃+r₂₃)/2. It has been found that using curved apertures as shown in FIGS. 15A-15B and FIG. 16 can reduce phase measurement error and/or provide more precise phase measurements.

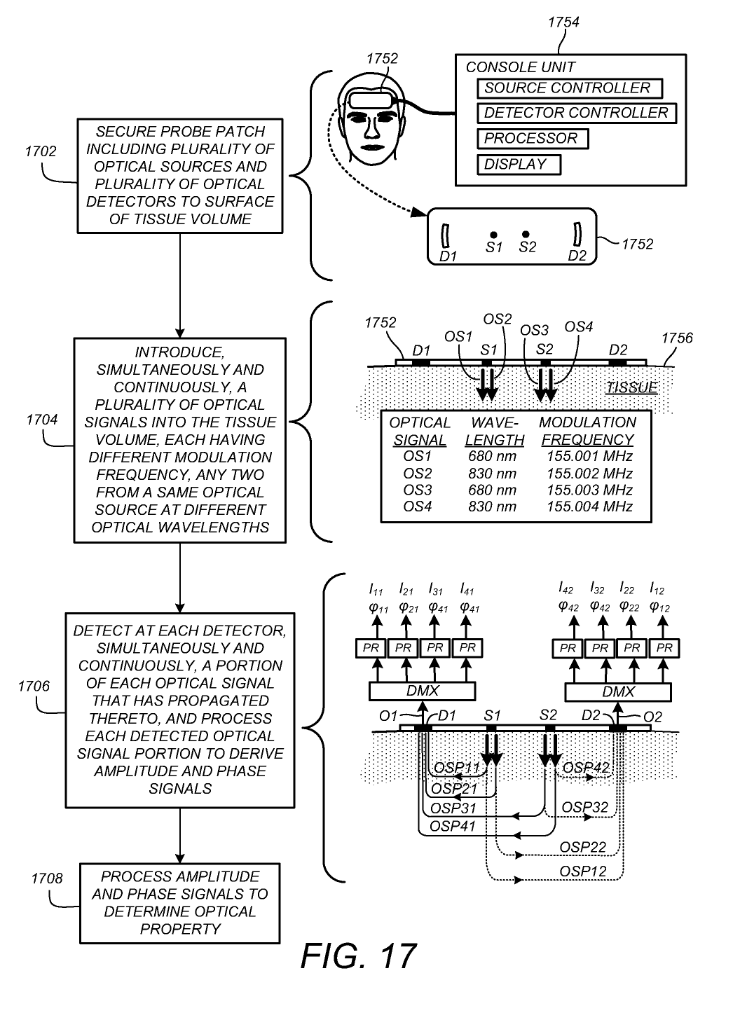

FIG. 17 illustrates NIRS monitoring of a tissue volume of a patient according to a preferred embodiment. At step 1702, a probe patch containing a plurality of optical sources and a plurality of optical detectors is secured to a surface of the tissue volume. Illustrated in FIG. 17 is a probe patch 1752 as secured to the forehead of a patient for cerebral oximetry. The probe patch is optically, electrically, or electrooptically coupled to a console unit 1754 that includes a source controller, a detector controller, at least one processor, and a display that are collectively configured and/or programmed to achieve the functionalities described herein. Preferably, as illustrated in FIG. 17, the probe patch 1752 includes curved-aperture detectors D1 and D2 similar to those described above with respect to FIGS. 15A, FIG. 15B, and/or FIG. 16.

At step 1704, the plurality of optical sources are operated to introduce, simultaneously and on a continuous basis throughout the patient monitoring session, a plurality of optical signals into the tissue volume, wherein each of the optical signals has a modulation frequency different than that of each other optical signal, and wherein any two of the optical signals that are introduced from a same one of the optical sources are at different optical wavelengths. Illustrated by way of example in FIG. 17 is a side view of the probe patch 1752 as secured to a surface 1756 of a tissue volume, wherein first and second optical signals (OS1 and OS2) are introduced into the tissue volume from source S1, and wherein third and fourth optical signals (OS3 and OS4) are introduced into the tissue volume from source S2, all signals being introduced simultaneously and continuously throughout the patient monitoring session. The optical signals OS1, OS2, OS3, and OS4 are all at different modulation frequencies, and any two of them emitted from a common optical source are at different optical wavelengths.

At step 1706, the plurality of optical detectors are operated to detect, simultaneously and on a continuous basis throughout the monitoring session, a portion of each of the optical signals that has propagated thereto, and the detected signal portions are processed to derive an amplitude signal and a phase signal associated therewith. Thus, for example, the first optical signal OS1 as introduced into the tissue volume by source S1 will have a first optical signal portion OSP11 that propagates to the detector D1, and will have a second optical signal portion OSP12 that propagates to the detector D2. Each detector will receive a portion of each of the optical signals OS1, OS2, OS3, and OS4 that has propagated thereto. For example, detector D1 will receive the optical signal portions OSP11, OSP21, OSP31, and OSP41, while detector D2 will receive the optical signal portions OSP12, OSP22, OSP32, and OSP42. Each detector will generate a first signal representative of an overall combination of the optical signal portions as received at that detector. For example, detector D1 will generate an overall signal O1 representative of the combination of the optical signal portions OSP11, OSP21, OSP31, and OSP41 received thereat.

Based on the different modulation frequencies of the optical signal portions OSP11, OSP21, OSP31, and OSP41 and using a circuit similar or analogous to that of FIG. 13, the console unit 1754 will demultiplex the first signal O1 into individual components corresponding to the optical signal portions OSP11, OSP21, OSP31, and OSP41, and then process these individual components to generate an amplitude signal and a phase signal associated with each of the optical signal portions. Likewise, based on the different modulation frequencies of the optical signal portions OSP12, OSP22, OSP32, and OSP42 and using a circuit similar or analogous to that of FIG. 13, the console unit 1754 will demultiplex the signal O2 into individual components corresponding to the optical signal portions OSP12, OSP22, OSP32, and OSP42, and then process these individual components to generate an amplitude signal and a phase signal associated with each of the optical signal portions.

Finally, at step 1708, the amplitude signals and a phase signals associated with the detected optical signal portions are processed to determine the optical property of the tissue volume. For one preferred embodiment, with reference generally to FIG. 9C-1 through FIG. 10, supra, the optical property can be computed at step 1708 according to the steps of: (i) for a nearer-spaced source-detector pair selected from the pluralities of optical sources and detectors, receiving the amplitude signals and phase signals for two corresponding optical signal portions having distinct wavelengths; (ii) for a farther-spaced source-detector pair selected from the pluralities of optical sources and detectors and including either the optical source or the optical detector of the nearer-spaced source-detector pair, receiving the amplitude signals and phase signals for two corresponding optical signal portions having distinct wavelengths; and (iii) processing the amplitude signals and phase signals corresponding to the nearer-spaced and farther-spaced source-detector pairs according to a slope-based phase modulation spectroscopy (PMS) algorithm to compute an absorption property and a scattering property relevant to at least a portion of the tissue volume.

Whereas many alterations and modifications of the present invention will no doubt become apparent to a person of ordinary skill in the art after having read the foregoing description, it is to be understood that the particular embodiments shown and described by way of illustration are in no way intended to be considered limiting. By way of example, although 100% or “full” duty cycle operation is particularly advantageous in the context of PMS (phase modulated spectrophotometry) systems, the scope of the preferred embodiments can also include CWS (continuous wave spectrophotometry) systems. For CWS schemes, even though phases are not measured, there is usually some modulation of the NIR signals performed to avoid 1/f effects, with typical modulation frequencies being on the order of 25 kHz. For these cases, the different source port/wavelength pairs can simply be modulated at distinct frequencies of 25 kHz, 26 kHz, 27 kHz, etc., with the overall received signal at each detector being separated into individual received signals based on these different frequencies.

By way of further example, although 100% or “full” duty cycle provides the most increase in signal to noise performance, it is also within the scope of the preferred embodiments to provide a less-than-full duty cycle system in which more than one, but fewer than all, of the “NM” different source port/wavelength pairs are emitting simultaneously. For example, a first half of the source port/wavelength pairs can simultaneously emit only during the first half of the acquisition cycle T_{A}, and a second half of the source port/wavelength pairs can simultaneously emit only during the second half of the acquisition cycle T_{A}. Such less-than-full duty cycle strategies could provide for relaxed demodulation/filtering hardware requirements and/or improved channel separation, while still providing for appreciably significant increases in signal to noise performance over the example of FIG. 11.

By way of still further example, one or more of the preferred embodiments supra are readily applicable for improving the signal to noise performance of NIRS monitoring systems that employ more than one “base” modulation frequency. The preferred embodiment of FIG. 12, for example, involved a single “base” modulation frequency of 155 MHz. Some known proposals for NIR spectrophotometric monitoring, however, are based on the use of multiple “base” modulation frequencies, such as that disclosed in the commonly assigned U.S. Pat. No. 7,551,950, in which two modulation frequencies (120 MHz and 150 MHz) are used in one of its examples. In such case, there is provided in accordance with one preferred embodiment a 50% duty cycle system, wherein all source port/wavelength pairs simultaneously emit at distinct frequencies around 120 MHz (e.g., 120.001 MHz, 120.002 MHz, 120.003 MHz, etc.) during the first half of the acquisition cycle, and then all source port/wavelength pairs simultaneously emit at distinct frequencies around 150 MHz (e.g., 150.001 MHz, 150.002 MHz, 150.003 MHz, etc.) during the second half of the acquisition cycle. Alternatively, there is provided in accordance with another preferred embodiment a 100% duty cycle system in which all source port/wavelength pairs emit simultaneously at all modulation frequencies (e.g., 120.001 MHz, 120.002 MHz, 120.003 MHz . . . , 150.001 MHz, 150.002 MHz, 150.003 MHz, etc.). Accordingly, it can be readily seen that the preferred embodiments are applicable across a wide variety of different NIRS implementations. Among other advantages, the improved signal to noise performance provided according to one or more preferred embodiments provides an ability to increase penetration depths in the non-invasive NIRS monitoring of crucial deep-layer tissue structures such as the human brain.

By way of even further example, in one preferred embodiment an NIR cerebral oximetry system is provided using the full-duty-cycle aspects and/or the curved aperture-shape aspects of one or more preferred embodiments supra in conjunction with the deep-layer-specific monitoring methods of the commonly assigned U.S. Ser. No. 12/815,696, supra. By way of even further example, there can be provided in an alternative preferred embodiment a scenario in which a same source is emitting at two different wavelengths simultaneously, wherein the modulation frequency for the two wavelengths is also identical. For this case, each detector port can be provided with a wavelength separation filter (e.g., a filter that passes light at 680 nm and reflects light at 830 nm) that separates the two optical signals based on optical wavelength, and then proceeds to separately demodulate those two optical signals. Therefore, reference to the details of the embodiments are not intended to limit their scope, which is limited only by the scope of the claims set forth below.

What is claimed is:

1. A method for non-invasive spectrophotometric monitoring of an optical property of a tissue volume during a patient monitoring session, comprising:
securing a plurality of optical sources and a plurality of optical detectors to a surface of the tissue volume;
operating said plurality of optical sources to introduce, simultaneously and on a continuous basis throughout the patient monitoring session, a plurality of optical signals into the tissue volume, wherein each said optical signal has a modulation frequency different than that of each other optical signal, and wherein any two of said optical signals that are introduced from a same one of the optical sources are at different optical wavelengths;
operating each of said plurality of optical detectors to detect, simultaneously and on a continuous basis throughout the monitoring session, a portion of each said optical signal that has propagated thereto, and processing each of said detected optical signal portions to derive an amplitude signal and a phase signal associated therewith; and
processing the amplitude signals and phase signals associated with said detected optical signal portions to determine the optical property of the tissue volume.

2. The method of claim 1, wherein said operating each of said plurality of optical detectors to detect said optical signal portions comprises:
receiving a first signal representative of an overall combination of said optical signal portions as received at that optical detector; and
demultiplexing said first signal into individual components according to the respective modulation frequencies of said optical signal portions.

3. The method of claim 2, wherein said determining the optical property of the tissue volume comprises:
for a nearer-spaced source-detector pair selected from said pluralities of optical sources and detectors, receiving the amplitude signals and phase signals for two corresponding optical signal portions having distinct wavelengths;
for a farther-spaced source-detector pair selected from said pluralities of optical sources and detectors and including either the optical source or the optical detector of the nearer-spaced source-detector pair, receiving the amplitude signals and phase signals for two corresponding optical signal portions having distinct wavelengths; and
processing said amplitude signals and phase signals corresponding to said nearer-spaced and farther-spaced source-detector pairs according to a slope-based phase modulation spectroscopy (PMS) algorithm to compute an absorption property and a scattering property relevant to at least a portion of the tissue volume.

4. The method of claim 2, wherein said optical signal portions each have an optical wavelength in the range of 500 nm-1000 nm, wherein said modulation frequencies are each greater than 100 MHz, and wherein said modulation frequencies differ from each other by less than 100 kHz.

5. The method of claim 1, wherein:
said patient monitoring session includes a calibration interval and a monitoring interval, said monitoring interval being subsequent to said calibration interval;
said plurality of optical sources includes a first optical source and a second optical source, and said plurality of optical detectors includes a first optical detector and a second optical detector;
said optical signal portions include a first pair of optical signal portions each propagated through the tissue volume between said first optical source and said first optical detector and having first and second respective wavelengths;
said optical signal portions include a second pair of optical signal portions each propagated through the tissue volume between said second optical source and said first optical detector and having said first and second respective wavelengths;
said optical signal portions include a third pair of optical signal portions each propagated through the tissue volume between said first optical source and said second optical detector and having said first and second respective wavelengths;
said optical signal portions include a fourth pair of optical signal portions each propagated through the tissue volume between said second optical source and said second optical detector and having said first and second respective wavelengths;
said first, second, third, and fourth pairs of optical signal portions as detected during said calibration interval are processed to compute at least one algorithm compensation that causes a first result related to said optical property based on said first and second detected pairs of optical signal portions to be substantially equal to a second result related to said optical property based on said third and fourth detected pairs of optical signal portions; and
said first, second, third, and fourth pairs of optical signal portions as detected during said monitoring interval are processed in conjunction with said at least one algorithm compensation to compute a monitoring result for the optical property of the tissue volume.

6. The method of claim 5, said first and second pairs of optical signal portions corresponding to a first subregion of the tissue volume, said third and fourth pairs of optical signal portions corresponding to a second subregion of the tissue volume that is at least partially non-overlapping with said first subregion, wherein said computing said at least one algorithm compensation comprises:
(i) computing at least one error factor associated with at least one non-ideality of said optical sources and/or detectors to which a difference in said first and second results would be attributable if the optical property was known to be spatially homogenous throughout said first and second subregions during said calibration interval; and
(ii) determining at least one compensation factor associated with said at least one error factor that causes said first and second results to be substantially equal for said calibration interval.

7. The method of claim 6, wherein said at least one non-ideality is associated with one or more of intensity of the optical sources, sensitivity of the optical detectors, coupling efficiency of light from the optical sources into the tissue volume, and coupling efficiency of light from the tissue volume to said optical detectors.

8. The method of claim 1, said optical sources and detectors including a first optical source and a first optical detector, said optical sources and detectors being positioned on a wearable patch secured to the surface of the tissue volume, wherein:
said first optical detector includes a first aperture formed in a tissue-facing surface of the wearable patch, the first aperture including a central area, a first edge positioned nearer to the first optical source than the central area, and a second edge positioned farther from the first optical source than the central area; and
said first and second edges of said first aperture are each curved concavely toward said first optical source.

9. The method of claim 8, wherein said first and second edges of said first aperture are each curved concavely toward said first optical source with a radius of curvature corresponding to a distance between said first optical detector and said first optical source.

10. The method of claim 8, said optical sources and detectors further including a second optical source and a second optical detector positioned on said wearable patch, wherein:
said wearable patch is generally elongate and includes first and second ends and a center region therebetween;
said first and second optical detectors are positioned near said first and second ends, respectively, and said first and second optical sources are positioned near said center region;
said second optical detector includes a second aperture formed in said tissue-facing surface and including a central area, a first edge positioned nearer to the first optical source than the central area, and a second edge positioned farther from the first optical source than the central area; and
said first and second edges of said second aperture are each curved concavely toward said first optical source.

11. The method of claim 10, wherein each of said first and second edges for each of said first and second apertures is curved concavely toward the center of the wearable patch with a radius of curvature corresponding to an average distance between that aperture and said first and second optical sources.

12. An apparatus for non-invasive spectrophotometric monitoring of an optical property of a tissue volume of a patient during a patient monitoring session, comprising:
a probe patch wearable on a surface of the tissue volume, the probe patch comprising a plurality of optical sources and a plurality of optical detectors, the probe patch being configured to maintain each of said optical sources and each of said optical detectors in secured contact with the surface of the tissue volume throughout the patient monitoring session;
a source controller coupled to each of said plurality of optical sources, said source controller being configured to cause said plurality of optical sources to introduce, simultaneously and on a continuous basis throughout the patient monitoring session, a plurality of optical signals into the tissue volume, each said optical signal having a modulation frequency different than that of each other optical signal, wherein any two of said optical signals that are introduced from a same one of the optical sources are at different optical wavelengths;
a detector controller coupled to each of said plurality of optical detectors, said detector controller being configured to cause each of said plurality of optical detectors to detect, simultaneously and on a continuous basis throughout the monitoring session, a portion of each said optical signal that has propagated thereto; and
at least one processor configured to process each of the detected optical signal portions to derive an amplitude signal and a phase signal associated therewith, the at least one processor being further configured to process the amplitude signals and phase signals associated with the detected optical signal portions to determine the optical property of the tissue volume.

13. The apparatus of claim 12, wherein said detector controller is configured to cause each of said plurality of detectors to receive a combination of the optical signal portions incident thereon and to demultiplex said combination into individual components according to the respective modulation frequencies of the incident optical signal portions.

14. The apparatus of claim 13, wherein said at least one processor determines the optical property of the tissue volume according to the steps of:
for a nearer-spaced source-detector pair selected from said pluralities of optical sources and detectors, receiving the amplitude signals and phase signals for two corresponding optical signal portions having distinct wavelengths;
for a farther-spaced source-detector pair selected from said pluralities of optical sources and detectors and including either the optical source or the optical detector of the nearer-spaced source-detector pair, receiving the amplitude signals and phase signals for two corresponding optical signal portions having distinct wavelengths; and
processing said amplitude signals and phase signals corresponding to said nearer-spaced and farther-spaced source-detector pairs according to a slope-based phase modulation spectroscopy (PMS) algorithm to compute an absorption property and a scattering property relevant to at least a portion of the tissue volume.

15. The apparatus of claim 13, wherein said optical signal portions each have an optical wavelength in the range of 500 nm-1000 nm, wherein said modulation frequencies are each greater than 100 MHz, and wherein said modulation frequencies differ from each other by less than 100 kHz.

16. The apparatus of claim 12, said optical sources and detectors including a first optical source and a first optical detector, said first optical detector including a first aperture formed in a tissue-facing surface of the wearable patch, the first aperture including a central area, wherein:
said first aperture includes first edge positioned nearer to the first optical source than the central area and a second edge positioned farther from the first optical source than the central area; and
said first and second edges of said first aperture are each curved concavely toward said first optical source.

17. The apparatus of claim 16, wherein said first and second edges of said first aperture are each curved concavely toward said first optical source with a radius of curvature corresponding to a distance between said first optical detector and said first optical source.

18. The apparatus of claim 16, said optical sources and detectors further including a second optical source and a second optical detector positioned on said wearable patch, wherein:
said wearable patch is generally elongate and includes first and second ends and a center region therebetween;
said first and second optical detectors are positioned near said first and second ends, respectively, and said first and second optical sources are positioned near said center region;
said second optical detector includes a second aperture formed in said tissue-facing surface, said second aperture including a central area, a first edge positioned nearer to the first optical source than the central area, and a second edge positioned farther from the first optical source than the central area; and
said first and second edges of said second aperture are each curved concavely toward said first optical source.

19. An apparatus for non-invasive spectrophotometric monitoring of an optical property of a tissue volume of a patient during a patient monitoring session, comprising:
a probe patch wearable on a surface of the tissue volume of the patient;
a first optical source and a first optical detector disposed on said probe patch, the probe patch being configured to maintain said first optical source and said first optical detector in secured contact with the surface of the tissue volume throughout the patient monitoring session;
wherein said first optical detector includes a first aperture formed in a tissue-facing surface of the wearable patch, the first aperture including a central area, a first edge positioned nearer to the first optical source than the central area, and a second edge positioned farther from the first optical source than the central area;
and wherein said first and second edges of said first aperture are each curved concavely toward said first optical source.

20. The apparatus of claim 19, wherein said first and second edges of said first aperture are each curved concavely toward said first optical source with a radius of curvature corresponding to a distance between said first optical detector and said first optical source.

21. The apparatus of claim 20, further including a second optical source and a second optical detector positioned on said wearable patch, wherein:
said wearable patch is generally elongate and includes first and second ends and a center region therebetween;
said first and second optical detectors are positioned near said first and second ends, respectively, and said first and second optical sources are positioned near said center region;
said second optical detector includes a second aperture formed in said tissue-facing surface, said second aperture including a central area, a first edge positioned nearer to the first optical source than the central area, and a second edge positioned farther from the first optical source than the central area; and
said first and second edges of said second aperture are each curved concavely toward said first optical source.

22. The apparatus of claim 19, further comprising a plurality of optical sources including said first optical source and a plurality optical detectors including said first optical detector, the apparatus further comprising:
a source controller coupled to each of said plurality of optical sources, said source controller being configured to cause said plurality of optical sources to introduce, simultaneously and on a continuous basis throughout the patient monitoring session, a plurality of optical signals into the tissue volume, each said optical signal having a modulation frequency different than that of each other optical signal, wherein any two of said optical signals that are introduced from a same one of the optical sources are at different optical wavelengths;
a detector controller coupled to each of said plurality of optical detectors, said detector controller being configured to cause each of said plurality of optical detectors to detect, simultaneously and on a continuous basis throughout the monitoring session, a portion of each said optical signal that has propagated thereto; and
at least one processor configured to process each of the detected optical signal portions to derive an amplitude signal and a phase signal associated therewith, the at least one processor being further configured to process the amplitude signals and phase signals associated with the detected optical signal portions to determine the optical property of the tissue volume.

23. The apparatus of claim 22, wherein said detector controller is configured to cause each of said plurality of detectors to receive a combination of the optical signal portions incident thereon and to demultiplex said combination into individual components according to the respective modulation frequencies of the incident optical signal portions.

24. The apparatus of claim 23, wherein said at least one processor determines the optical property of the tissue volume according to the steps of:
for a nearer-spaced source-detector pair selected from said pluralities of optical sources and detectors, receiving the amplitude signals and phase signals for two corresponding optical signal portions having distinct wavelengths;
for a farther-spaced source-detector pair selected from said pluralities of optical sources and detectors and including either the optical source or the optical detector of the nearer-spaced source-detector pair, receiving the amplitude signals and phase signals for two corresponding optical signal portions having distinct wavelengths; and
processing said amplitude signals and phase signals corresponding to said nearer-spaced and farther-spaced source-detector pairs according to a slope-based phase modulation spectroscopy (PMS) algorithm to compute an absorption property and a scattering property relevant to at least a portion of the tissue volume.

25. The apparatus of claim 23, wherein said optical signal portions each have an optical wavelength in the range of 500 nm-1000 nm, wherein said modulation frequencies are each greater than 100 MHz, and wherein said modulation frequencies differ from each other by less than 100 kHz.
